(19) United States
(12) Patent Application Publication
(10) Pub. No.: US 2026/0209204 A1
(43) Pub. Date: Jul. 23, 2026

(54) HETEROCYCLIC VAV1 DEGRADERS

(71) Applicant: INNOCARE PHARMA INC., Wilmington, DE (US)

(72) Inventors: Yingxiang GAO, Beijing (CN); Xiangyang CHEN, Beijing (CN)

(21) Appl. No.: 19/571,267

(22) Filed: Mar. 18, 2026

Related U.S. Application Data
(63) Continuation of application No. PCT/CN2025/092359, filed on Apr. 30, 2025.

(30) Foreign Application Priority Data
Sep. 2, 2024 (CN) 202411226410.8
Dec. 5, 2024 (CN) 202411784444.9

(51) Int. Cl. C07D 401/10; C07D 401/14; C07D 405/14

(52) U.S. Cl. CPC C07D 401/10; C07D 401/14; C07D 405/14

(57) ABSTRACT

The present invention relates to heterocyclic compounds or their pharmaceutically acceptable salts as VAV1 protein degraders. Specifically, the invention relates to compounds of Formula I and their pharmaceutically acceptable salts thereof. The invention also relates to methods for preparing the compounds of the present invention. The compounds of the present invention can be used for the treatment and/or prevention of VAV1-mediated diseases, including autoimmune and inflammatory diseases, and cancers.

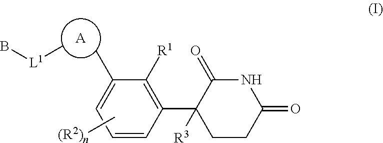

### CROSS-REFERENCE TO RELATED APPLICATION(S)

This application is a continuation of PCT/CN2025/092359, filed Apr. 30, 2025; which claims priority to Chinese Application Nos. 202411226410.8, filed Sep. 2, 2024, and 202411784444.9, filed Dec. 5, 2024. The contents of the above-identified applications are incorporated herein by reference in their entirety.

### TECHNICAL FIELD

The present invention relates to heterocyclic compounds of Formula I or their pharmaceutically acceptable salts thereof as VAV1 protein degraders. The invention also relates to methods for preparing compounds of Formula I. The invention further relates to the use of compounds of Formula I or their pharmaceutically acceptable salts thereof for the treatment and/or prevention of diseases mediated by VAV1, including autoimmune and inflammatory diseases, and cancers.

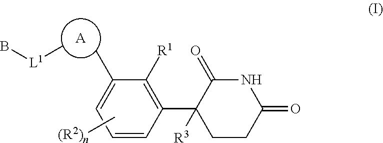

### BACKGROUND

VAV1 is a multi-domain protein with dual functions, acting as both a GEF (guanine nucleotide exchange factor) and a scaffolding protein involved in GEF-independent signaling pathways. The results from VAV1−/− mice indicate that VAV1 plays a critical role in the function of lymphocytes, such as T and B cells, as well as in antigen receptor signaling (Fischer, K. D. et al. Curr. Biol. 1998, 8, 554-562; Fujikawa, K. et al. J. Exp. Med. 2003, 198, 1595-1608). Therefore, targeted therapy against VAV1 has therapeutic potentials in the treatment of T and B cell-mediated autoimmune diseases, inflammation, and cancer.

### SUMMARY

However, due to the unique structure and function of VAV1, it is used to be considered as an undruggable target. Targeted protein degradation is a novel drug development strategy that facilitates the interaction between the ubiquitin-proteasome system (UPS) and disease-related proteins (referred to as “neo-substrates”) through small molecules (known as “molecular glues”), leading to the degradation of the neo-substrates. A clinically validated class of molecular glues is the glutarimide compounds, which bind to cereblon (CRBN) of the CRL4^{CRBN}-E3 ubiquitin ligase complex, inducing a change in the surface structure of the CRBN protein. This alteration promotes E3 ligase to recruit neo-substrates that are typically undruggable, leading to their ubiquitination by E2 and eventual recognition and degradation by 26S proteasome, thereby regulating the biological functions mediated by these neo-substrates. There is no clinical efficacy reported for degraders targeting VAV1.

The present invention relates to heterocyclic compounds according to Formula I and shows that compounds with such structures exhibit good activity in inducing VAV1 degradation.

### BRIEF DESCRIPTION OF THE DRAWINGS

FIG. 1 shows that daily oral dosing of compounds I-6, I-12, I-14, and I-20 at 1 mg/kg significantly alleviated EAE progression with less clinical scores in the treatment groups compared to those in the vehicle group.

FIG. 2 shows that daily oral dosing of compounds I-6, I-12, I-14, and I-20 at 1 mg/kg did not have significant weight loss in the treatment groups compared to those in the vehicle group.

### DETAILED DESCRIPTION

### Definition

Unless otherwise stated, the following terms used in this application have the meanings described below.

“C_{x-y}” refers to the range of carbon atoms, where x and y are both integers. For example, C₃₋₈ cycloalkyl indicates a cycloalkyl group with 3-8 carbon atoms, that is, a cycloalkyl group with 3, 4, 5, 6, 7, or 8 carbon atoms. It should also be understood that “C₃₋₈” includes any sub-range within it, such as C₃₋₇, C₃₋₆, C₄₋₇, C₄₋₆, C₅₋₆, etc.

“Alkyl” refers to a saturated straight or branched hydrocarbon group containing 1 to 20 carbon atoms, such as 1 to 8 carbon atoms, 1 to 6 carbon atoms, or 1 to 4 carbon atoms. Non-limiting examples of alkyl include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, etc.

“Alkylene” refers to a divalent saturated hydrocarbon group containing 1 to 20 carbon atoms, such as a straight or branched chain with 1 to 6 carbon atoms, 1 to 4 carbon atoms, etc. Non-limiting examples of alkylene include —CH₂—, —CH(CH₃)—, —CH₂CH₂—, —CH₂CH₂CH₂—, —(CH₃)C(CH₃)—, —CH₂CH₂CH₂CH₂—, —CH₂CH(CH₃)CH₂—, etc.

“Cycloalkyl or cycloalkane” refers to a saturated cyclic hydrocarbon group containing 3 to 14 carbon ring atoms, which can be monocyclic, typically containing 3 to 8, 3 to 7, or 3 to 6 carbon ring atoms. Non-limiting examples of monocyclic cycloalkyl include cyclopropyl, cyclobutyl, cyclopentyl, cyclohexyl, cycloheptyl, etc. They can also be fused, bridged, or spirocyclic bicyclic or tricyclic, such as decahydronaphthyl, bicyclo[2.2.2]octane, spiro[3.3]heptane, etc.

“Cycloalkylene” refers to a divalent saturated carbocyclic group having 3 to 14 carbon ring atoms, for example, a structure as

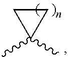

where n is an integer of 1 or greater than 1, typically n as 1 to 12. Non-limiting examples of cycloalkylene include cyclopropene, cyclobutene, cyclopentene, cyclohexene, etc.

“Heterocyclic or heterocycle” refers to a saturated or partially unsaturated monocyclic or polycyclic group, which include 3 to 20 ring atoms, for example, 3 to 14, 3 to 12, 3 to 10, 3 to 8, 3 to 6, or 5 to 6 ring atoms, where one or more ring atoms are selected from nitrogen, oxygen, or S(O)ₘ (where m is an integer from 0 to 2), and the remaining ring atoms are carbon. Preferably, it includes 3 to 12 ring atoms, 3 to 10 ring atoms, 4 to 7 ring atoms, 4 to 6 ring atoms, where 1 to 4 are heteroatoms, 1 to 3 are heteroatoms, or 1 to 2 are heteroatoms. Non-limiting examples of monocyclic heterocyclic include oxetanyl, tetrahydrofuranyl, tetrahydropyranyl, azetidinyl, pyrrolidinyl, piperidinyl, piperazinyl, morpholinyl, thiomorpholinyl, 1,4-oxazepanyl, pyrrolidinonyl, piperidinonyl, 1,1-dioxothiomorpholinyl, etc. Polycyclic heterocyclics include fused, bridged, or spiro polycyclic heterocycles, such as octahydrocyclopenta[c]pyrrole, octahydropyrrolo[1,2-a]pyrazine, 3,8-diazabicyclo[3.2.1]octane, 5-azaspiro[2.4]heptane, 2-oxa-7-azaspiro[3.5]nonane, etc.

“Aryl or aryl ring” refers to an aromatic monocyclic or fused polycyclic group containing 6 to 14 carbon atoms, preferably 6 to 10 members, such as phenyl and naphthyl, more preferably phenyl. The aryl ring can be fused to a heteroaryl, heterocyclic, or cycloalkyl ring, where the ring connected to the parent structure is the aryl ring. Non-limiting examples include:

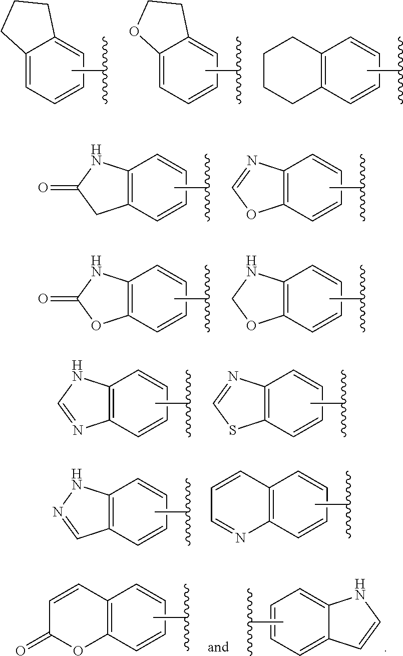

“Heteroaryl or heteroaryl ring” refers to a heteroaromatic system containing 5 to 14 ring atoms, where 1 to 4 ring atoms are selected from heteroatoms including oxygen, sulfur, and nitrogen. Heteroaryl is preferably 5 to 10-membered, more preferably 5-membered or 6-membered, such as furyl, thienyl, pyridyl, pyrrolyl, pyrimidinyl, pyrazinyl, pyrazolyl, imidazolyl, tetrazolyl, oxazolyl, isoxazolyl, thiazolyl, isothiazolyl, quinolinyl, isoquinolinyl, indolyl, isoindolyl, etc. The heteroaryl ring can be fused to an aryl, heterocyclic, or cycloalkyl ring. Non-limiting examples include:

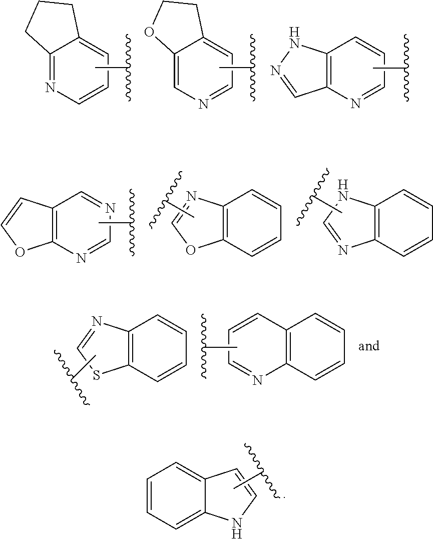

“Halogen” refers to fluorine, chlorine, bromine or iodine.

“Cyano” refers to —CN.

“Oxo” refers to ═O.

“Optional” means that the event or circumstance described subsequently may or may not occur. For example, the description “one or more hydrogens of phenyl are optionally substituted by halogen” includes scenarios where one or more hydrogens of phenyl are substituted by halogen and where none is substituted by halogen.

“Substitution” refers to one or more hydrogen atoms in a group, preferably 1 to 5, for example, 1 to 3 hydrogen atoms, being independently replaced by a corresponding number of substituents. Substituents are only located at possible chemical positions understood by those skilled in the art. For example, an amino or hydroxyl group with free hydrogen may be unstable when connected to a carbon atom having an unsaturated bond (such as an alkene). Substituents include but are not limited to halogen, cyano, nitro, hydroxy, amino, oxo, —SF₅, C₁₋₄ alkyl, C₃₋₇ cycloalkyl, 4- to 7-membered heterocyclyl, phenyl, 5- to 6-membered heteroaryl, etc.

“Isomer” refers to a compound that has the same molecular formula but differs in the bonding positions or spatial arrangements of their atoms. Isomers with different spatial arrangements of atoms are called “stereoisomers”. Stereoisomers include optical isomers, geometric isomers, and conformational isomers.

The compounds of the present invention can exist in the form of optical isomers. Optical isomers include enantiomers and diastereomers. Enantiomers refer to two stereoisomers that are non-superimposable and are mirror images of each other. A racemic mixture or racemate refers to a chiral molecule mixture containing equal amounts of left-handed and right-handed enantiomers. Diastereomers refer to two stereoisomers that are non-superimposable and are not mirror images of each other. When the optical isomer is a single isomer and its absolute configuration is determined, it is referred to as “R” or “S” isomer based on the configuration of the substituents on the chiral atom. When the absolute configuration is not determined, it is referred to as the (+) or (−) isomer based on its measured optical rotation value. The methods for preparing and separating optical isomers are known in the art.

The compounds of the present invention may also have geometric isomers with substituents distributed differently around carbon-carbon double bonds, carbon-nitrogen double bonds, cycloalkyl, or heterocyclic group. Substituents around carbon-carbon double bonds or carbon-nitrogen bonds are designated as Z or E configurations, while substituents around cycloalkyl or heterocyclic group are designated as cis or trans configurations.

The compounds of the present invention may also exhibit tautomerism, such as keto-enol tautomerism.

The present invention includes any tautomeric or stereoisomeric forms and their mixtures and is not limited to any one tautomeric or stereoisomeric form used in the naming or chemical structural formula of the compound.

“Isotopes” refer to all isotopes of atoms that appear in the compounds of this invention. Isotopes include those atoms having the same atomic number but different mass numbers. Examples of isotopes suitable for incorporation into the compounds of this invention are hydrogen, carbon, nitrogen, oxygen, phosphorus, sulfur, fluorine, and chlorine, specifically but not limited to ²H (D), ³H, ¹³C, ¹⁴C, ¹⁵N, ¹⁷O, ¹⁸O, ³¹P, ³²P, ³⁵S, ¹⁸F and ³⁶Cl. The isotopically labeled compounds of this invention can generally be prepared by conventional techniques known to those skilled in the art or by using appropriately isotopically labeled reagents in place of non-isotopically labeled reagents in methods like those described in the examples. Such compounds have various potential uses, such as standards and reagents in determining biological activity. In terms of stable isotopes, such as deuterium ²H (D), ¹³C, and ¹⁵N, compounds containing such isotopes have the potential to alter biological, pharmacological or pharmacokinetic properties. Deuterium ²H (D) is a preferred isotope of this invention, for example, hydrogens in methyl, methylene, or methyne can be replaced by deuterium.

The compounds of the present invention can be administered in the form of prodrugs. “Prodrug” refers to a derivative that is converted into biologically active compound of the present invention under physiological conditions in vivo, for example, through oxidation, reduction, hydrolysis (each of which occurs with or without the participation of enzymes). Examples of prodrugs include the following compounds of the present invention in which an amino is acylated, alkylated or phosphorylated, for example, eicosanoyl amino, alanyl amino and pivaloyloxymethyl amino, or a hydroxyl is acylated, alkylated, phosphorylated or converted into borate, for example, acetoxy, palmitoyloxy, pivaloyloxy, succinyloxy, fumaroyloxy and alanyloxy, or a carboxyl is esterified or amidated, or a thiol is conjugated with a carrier molecule that selectively delivers the drug to the target and/or to the cytosol of cells. These compounds can be prepared from the compounds of the present invention by known methods.

“Pharmaceutically acceptable salts” refer to salts made from the compounds of the present invention containing one or more acidic and basic groups with pharmaceutically acceptable bases or acids, including inorganic bases or acids and organic bases or acids. Therefore, compounds of the present invention containing acidic groups can exist in form of salts, such as alkali metal salts, alkaline earth metal salts, or ammonium salts. More specific examples of such salts include sodium salts, potassium salts, calcium salts, magnesium salts, or salts with ammonia or organic amines, such as ethylamine, ethanolamine, triethanolamine, or amino acids. Compounds of the present invention containing basic groups can exist in form of inorganic or organic acid salts. Examples of suitable acids include hydrochloric acid, hydrobromic acid, phosphoric acid, sulfuric acid, nitric acid, methanesulfonic acid, p-toluenesulfonic acid, naphthalenedisulfonic acid, oxalic acid, acetic acid, tartaric acid, lactic acid, salicylic acid, benzoic acid, formic acid, propionic acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid, and other acids known to those skilled in the art. If compound of the present invention contains both acidic and basic groups in the molecule, the present invention includes zwitterions or internal salts in addition to the mentioned salt forms. Each salt can be obtained by conventional methods known to those skilled in the art, such as by mixing the compound with an organic or inorganic acid or base in a solvent or dispersant, or by anion or cation exchange with other salts.

“Pharmaceutical composition” refers to a composition containing one or more compounds of the present invention or their pharmaceutically acceptable salts, stable isotope derivatives, isomers, prodrugs and mixtures thereof, as well as other components, such as pharmaceutically acceptable carriers and excipients.

When “compounds” are mentioned in the present invention, all compound forms are included, such as pharmaceutically acceptable salts, stable isotope derivatives, isomers, prodrugs, and mixtures thereof.

“Therapeutically effective amount” refers to the amount of the compound of the present invention that can effectively degrade VAV1, for the treatment or prevention of diseases mediated by VAV1.

“Patients” refer to mammals, preferably humans.

The present invention relates to compounds as VAV1 degraders, having a structure of Formula I, or their pharmaceutically acceptable salts, stable isotope derivatives, isomers and prodrugs thereof:

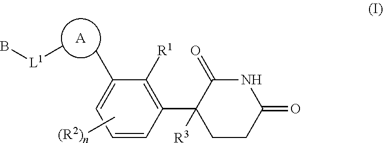

wherein:
Ring A is

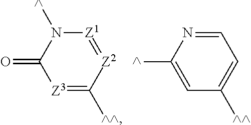

or 5-membered heteroaryl ring, where \hat{ } indicates the bond connected to L¹, \hat{ }\hat{ } indicates the bond connected to the benzene ring, and one or more hydrogens of the pyridine and 5-membered heteroaryl ring are optionally substituted by R⁴;
B is 5- to 10-membered heteroaryl, C₆₋₁₀ aryl, C₁₋₆ alkyl, C₃₋₈ cycloalkyl or 4- to 10-membered heterocyclyl, where one or more hydrogens of the heteroaryl, aryl, alkyl, cycloalkyl and heterocyclyl are optionally substituted by R⁵;
Z¹, Z² and Z³ are each independently CR^{4′} or N;
L¹ is —C₁₋₆ alkylene-, bond, —C₃₋₆ cycloalkylene-, —C(O)—, —O—, —S(O)₀₋₂—, —NR′—, *—OC₁₋₆ alkylene-**, *—S(O)₀₋₂C₁₋₆ alkylene-**, *—NR′C₁₋₆ alkylene-**, *—C₁₋₆ alkylene-O—**, *—C₁₋₆ alkylene-S(O)₀₋₂—**, *—C₁₋₆ alkylene-NR′—**, *—NR′C(O)—** or *—C(O)NR′—**, where * indicates L¹ connected to ring A, ** indicates L¹ connected to B, and one or more hydrogens of the alkylene and cycloalkylene are optionally substituted by D, halogen or C₁₋₆ alkyl;
R¹ is halogen, H, cyano, C₁₋₆ alkyl, C₃₋₈ cycloalkyl or —OC₁₋₆ alkyl, where one or more hydrogens of the alkyl are optionally substituted by D or halogen;
Each R² is independently H, halogen, cyano, —OR′, —NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl;
R³ is H, D, halogen, C₁₋₆ alkyl or fluorinated C₁₋₆ alkyl;
R^{4′} is H or R⁴;
R⁴ and R⁵ are each independently halogen, cyano, oxo, —OR′, —NR′R″, —COOH, —C(O)NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl;
R′ and R″ are each independently H, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OC₁₋₆ alkyl or C₁₋₆ alkyl; and
n is an integer from 1 to 3.

In one embodiment, ring A is

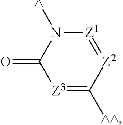

where \hat{ } indicates the bond connected to L¹, \hat{ }\hat{ } indicates the bond connected to the benzene ring, and Z¹, Z² and Z³ are each independently CH or N.

In one embodiment, ring A is

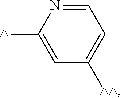

where \hat{ } indicates the bond connected to L¹, \hat{ }\hat{ } indicates the bond connected to the benzene ring.

In one embodiment, ring A is 5-membered heteroaryl containing 1-3 heteroatoms of nitrogen, oxygen and sulfur.

In one embodiment, B is 5- to 6-membered heteroaryl containing 1-3 heteroatoms of nitrogen, oxygen and sulfur, where one or more hydrogens of the heteroaryl are optionally substituted by halogen, cyano, oxo, —OR′, —NR′R″, C₁₋₆ alkyl, C₃₋₈ cycloalkyl or 4- to 8-membered heterocyclyl, where one or more hydrogens of the alkyl, cycloalkyl and heterocyclyl are further optionally substituted by D or halogen.

In one embodiment, L¹ is —C₁₋₆ alkylene-, where one or more hydrogens of the alkylene are optionally substituted by D, halogen or C₁₋₆ alkyl.

In one embodiment, L¹ is bond.

In one embodiment, L¹ is —O— or —OC₁₋₂ alkylene-.

In one embodiment, R¹ is halogen.

In one embodiment, R² is H.

In one embodiment, R³ is H or F.

In one embodiment, R′ and R″ are each H.

In some embodiments, the compounds of the present invention have Formula II:

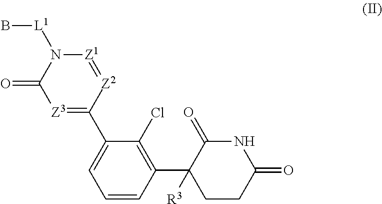

wherein:
B is 5- to 6-membered heteroaryl, phenyl or 4- to 10-membered heterocyclyl, where one or more hydrogens of the heteroaryl, phenyl and heterocyclyl are optionally substituted by R⁵;
Z¹, Z² and Z³ are each independently CR^{4′} or N;
L¹ is a —C₁₋₆ alkylene-, bond, *—C₂₋₆ alkylene-O—** or *—C₂₋₆ alkylene-NR′—**, where * indicates L¹ connected to the N atom of the heteroaryl ring, ** indicates L¹ connected to B, and one or more hydrogens of the alkylene are optionally substituted by D, halogen or C₁₋₆ alkyl;
R³ is H, D, halogen, C₁₋₆ alkyl or fluorinated C₁₋₆ alkyl;
R^{4′} is H or R⁴;
R⁴ and R⁵ are each independently halogen, cyano, oxo, —OR′, —NR′R″, —COOH, —C(O)NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl; and
R′ and R″ are each independently H, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OC₁₋₆ alkyl or C₁₋₆ alkyl.

In one embodiment, B is 5- to 6-membered heteroaryl containing 1-3 heteroatoms of nitrogen, oxygen and sulfur, where one or more hydrogens of the heteroaryl are optionally substituted by halogen, cyano, oxo, —OR′, —NR′R″, C₁₋₆ alkyl, C₃₋₈ cycloalkyl or 4- to 8-membered heterocyclyl, where one or more hydrogens of the alkyl, cycloalkyl and heterocyclyl are further optionally substituted by D or F.

In one embodiment, L¹ is —C₁₋₆ alkylene-, where one or more hydrogens of the alkylene are optionally substituted by D, halogen or C₁₋₆ alkyl.

In one embodiment, R³ is H or F.

In one embodiment, R″ is H.

In one embodiment, R′ and R″ are each H.

In some embodiments, the compounds of the present invention have Formula III:

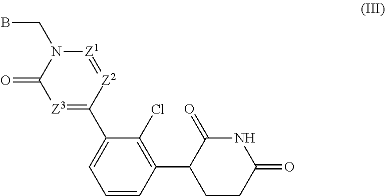

wherein:
B is 5- to 6-membered heteroaryl containing 1-3 heteroatoms of nitrogen, oxygen and sulfur, where one or more hydrogens of the heteroaryl are optionally substituted by R⁵;
Z¹, Z² and Z³ are each independently CH or N; and
R⁵ is halogen, cyano, oxo, —OR′, —NR′R″, —COOH, —C(O)NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl; and
R′ and R″ are each independently H, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OC₁₋₆ alkyl or C₁₋₆ alkyl.

In one embodiment, R⁵ is halogen, cyano, C₁₋₆ alkyl, C₃₋₈ cycloalkyl, —OC₁₋₆ alkyl or —OC₃₋₈ cycloalkyl, where one or more hydrogens of the alkyl are optionally substituted by D and F.

In one embodiment, R′ and R″ are each H.

In some embodiments, the compounds of the present invention have Formula IV:

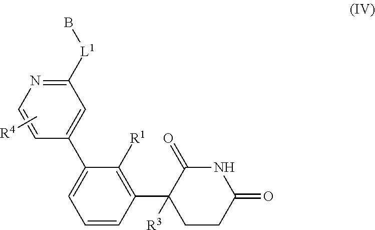

wherein:
B is 5- to 6-membered heteroaryl containing 1-3 heteroatoms of nitrogen, oxygen and sulfur or 4- to 10-membered heterocyclyl containing 1-2 heteroatoms of nitrogen, oxygen and sulfur, where one or more hydrogens of the heteroaryl and heterocyclyl are optionally substituted by R⁵;
L¹ is —C₁₋₆ alkylene-, bond, —O—, *—OC₁₋₆ alkylene-** or *—C₁₋₆ alkylene-O—**, where * indicates L¹ connected to pyridine, ** indicates L¹ connected to B, and one or more hydrogens of the alkylene are optionally substituted by D, halogen or —C₁₋₆ alkyl;
R¹ is halogen, H, cyano, C₁₋₆ alkyl, C₃₋₈ cycloalkyl or —OC₁₋₆ alkyl, where one or more hydrogens of the alkyl are optionally substituted by D or halogen;
R³ is H, D, halogen, C₁₋₆ alkyl or fluorinated C₁₋₆ alkyl;
R⁴ is H, halogen, C₁₋₆ alkyl or C₃₋₈ cycloalkyl, where one or more hydrogens of the alkyl are optionally substituted by D or halogen;
R⁵ is halogen, cyano, oxo, —OR′, —NR′R″, —COOH, —C(O)NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, C₁₋₆ alkyl or —OC₁₋₆ alkyl; and
R′ and R″ are each independently H, C₁₋₆ alkyl or C₃₋₈ cycloalkyl, where one or more hydrogens of the alkyl are optionally substituted by D or halogen.

In one embodiment, L¹ is —O—.

In one embodiment, L¹ is *—OC₁₋₆ alkylene-**, where * indicates L¹ connected to pyridine and ** indicates L¹ connected to B.

In one embodiment, R¹ is Cl.

In one embodiment, R³ is H.

In one embodiment, R⁴ is H.

In one embodiment, R⁵ is halogen, cyano, C₁₋₆ alkyl, C₃₋₈ cycloalkyl, —OC₁₋₆ alkyl or —OC₃₋₈ cycloalkyl.

The invention also relates to the following compounds, and their pharmaceutically acceptable salts, stable isotope derivatives, isomers, prodrugs and mixtures thereof:

| Compound | |
|---|---|
| # | Compound Structure and Nomenclature |
| I-1 | 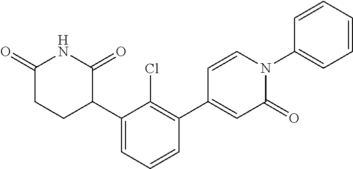 [SMILES: O=C1CCC(c2cccc(-c3ccn(-c4ccccc4)c(=O)c3)c2Cl)C(=O)N1] |
| | 3-(2-chloro-3-(2-oxo-1-phenyl-1,2-dihydropyridin-4-yl)phenyl)piperidine- |
| | 2,6-dione |
| I-2 | 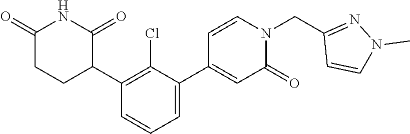 [SMILES: Cn1ccc(Cn2ccc(-c3cccc(C4CCC(=O)NC4=O)c3Cl)cc2=O)n1] |
| | 3-(2-chloro-3-(1-((1-methyl-1H-pyrazol-3-yl)methyl)-2-oxo-1,2- |
| | dihydropyridin-4-yl)phenyl)piperidine-2,6-dione |
| I-3 | 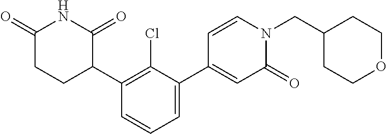 [SMILES: O=C1CCC(c2cccc(-c3ccn(CC4CCOCC4)c(=O)c3)c2Cl)C(=O)N1] |
| | 3-(2-chloro-3-(2-oxo-1-((tetrahydro-2H-pyran-4-yl)methyl)-1,2- |
| | dihydropyridin-4-yl)phenyl)piperidine-2,6-dione |
| I-4 | 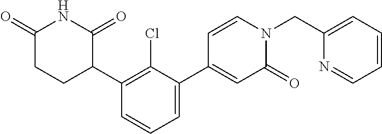 [SMILES: O=C1CCC(c2cccc(-c3ccn(Cc4ccccn4)c(=O)c3)c2Cl)C(=O)N1] |
| | 3-(2-chloro-3-(2-oxo-1-(pyridin-2-ylmethyl)-1,2-dihydropyridin-4- |
| | yl)phenyl)piperidine-2,6-dione |
| I-5 | 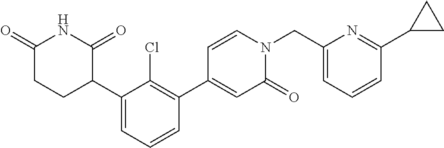 [SMILES: O=C1CCC(c2cccc(-c3ccn(Cc4cccc(C5CC5)n4)c(=O)c3)c2Cl)C(=O)N1] |
| | 3-(2-chloro-3-(1-((6-cyclopropylpyridin-2-yl)methyl)-2-oxo-1,2- |
| | dihydropyridin-4-yl)phenyl)piperidine-2,6-dione |
| I-6 | 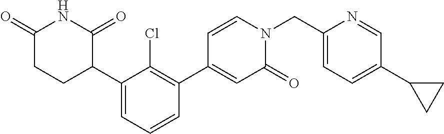 [SMILES: O=C1CCC(c2cccc(-c3ccn(Cc4ccc(C5CC5)cn4)c(=O)c3)c2Cl)C(=O)N1] |
| | 3-(2-chloro-3-(1-((5-cyclopropylpyridin-2-yl)methyl)-2-oxo-1,2- |
| | dihydropyridin-4-yl)phenyl)piperidine-2,6-dione |
| I-7 | 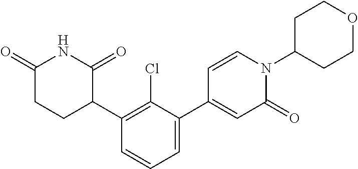 [SMILES: O=C1CCC(c2cccc(-c3ccn(C4CCOCC4)c(=O)c3)c2Cl)C(=O)N1] |
| | 3-(2-chloro-3-(2-oxo-1-(tetrahydro-2H-pyran-4-yl)-1,2-dihydropyridin-4- |
| | yl)phenyl)piperidine-2,6-dione |
| I-8 | 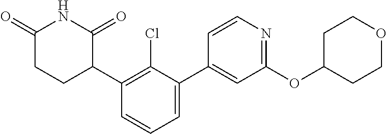 [SMILES: O=C1CCC(c2cccc(-c3ccnc(OC4CCOCC4)c3)c2Cl)C(=O)N1] |
| | 3-(2-chloro-3-(2-((tetrahydro-2H-pyran-4-yl)oxy)pyridin-4- |
| | yl)phenyl)piperidine-2,6-dione |
| I-9 | 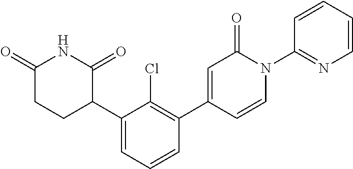 [SMILES: O=C1CCC(c2cccc(-c3ccn(-c4ccccn4)c(=O)c3)c2Cl)C(=O)N1] |
| | 3-(2-chloro-3-(2-oxo-2H-[1,2′-bipyridin]-4-yl)phenyl)piperidine-2,6-dione |
| I-10 | 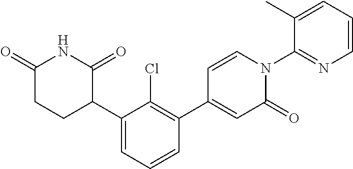 [SMILES: Cc1cccnc1-n1ccc(-c2cccc(C3CCC(=O)NC3=O)c2Cl)cc1=O] |
| | 3-(2-chloro-3-(3′-methyl-2-oxo-2H-[1,2′-bipyridin]-4-yl) |
| | phenyl)piperidine-2,6-dione |
| I-11 | 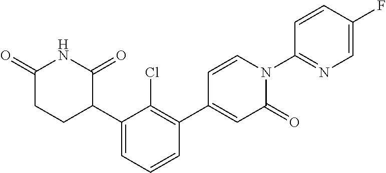 [SMILES: O=C1CCC(c2cccc(-c3ccn(-c4ccc(F)cn4)c(=O)c3)c2Cl)C(=O)N1] |
| | 3-(2-chloro-3-(5′-fluoro-2-oxo-2H-[1,2′-bipyridin]-4-yl)phenyl)piperidine- |
| | 2,6-dione |
| I-12 | 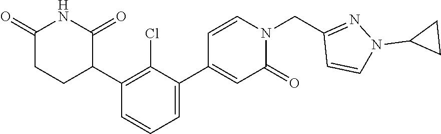 [SMILES: O=C1CCC(c2cccc(-c3ccn(Cc4ccn(C5CC5)n4)c(=O)c3)c2Cl)C(=O)N1] |
| | 3-(2-chloro-3-(1-((1-cyclopropyl-1H-pyrazol-3-yl)methyl)-2-oxo-1,2- |
| | dihydropyridin-4-yl)phenyl)piperidine-2,6-dione |
| I-13 | 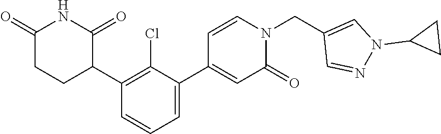 [SMILES: O=C1CCC(c2cccc(-c3ccn(Cc4cnn(C5CC5)c4)c(=O)c3)c2Cl)C(=O)N1] |
| | 3-(2-chloro-3-(1-((1-cyclopropyl-1H-pyrazol-4-yl)methyl)-2-oxo-1,2- |
| | dihydropyridin-4-yl)phenyl)piperidine-2,6-dione |
| I-14 | 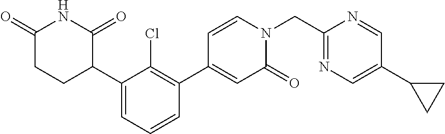 [SMILES: O=C1CCC(c2cccc(-c3ccn(Cc4ncc(C5CC5)cn4)c(=O)c3)c2Cl)C(=O)N1] |
| | 3-(2-chloro-3-(1-((5-cyclopropylpyrimidin-2-yl)methyl)-2-oxo-1,2- |
| | dihydropyridin-4-yl)phenyl)piperidine-2,6-dione |
| I-15 | 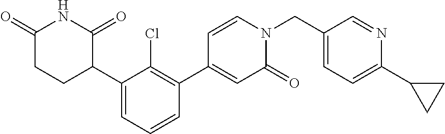 [SMILES: O=C1CCC(c2cccc(-c3ccn(Cc4ccc(C5CC5)nc4)c(=O)c3)c2Cl)C(=O)N1] |
| | 3-(2-chloro-3-(1-((6-cyclopropylpyridin-3-yl)methyl)-2-oxo-1,2- |
| | dihydropyridin-4-yl)phenyl)piperidine-2,6-dione |
| I-16 | 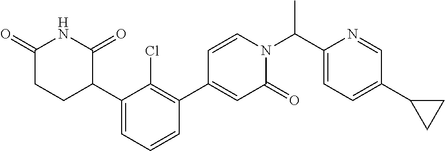 [SMILES: CC(c1ccc(C2CC2)cn1)n1ccc(-c2cccc(C3CCC(=O)NC3=O)c2Cl)cc1=O] |
| | 3-(2-chloro-3-(1-(1-(5-cyclopropylpyridin-2-yl)ethyl)-2-oxo-1,2- |
| | dihydropyridin-4-yl)phenyl)piperidine-2,6-dione |
| I-17 | 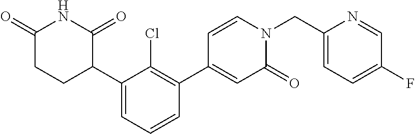 [SMILES: O=C1CCC(c2cccc(-c3ccn(Cc4ccc(F)cn4)c(=O)c3)c2Cl)C(=O)N1] |
| | 3-(2-chloro-3-(1-((5-fluoropyridin-2-yl)methyl)-2-oxo-1,2-dihydropyridin-4- |
| | yl)phenyl)piperidine-2,6-dione |
| I-18 | 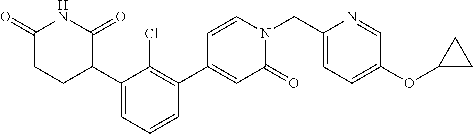 [SMILES: O=C1CCC(c2cccc(-c3ccn(Cc4ccc(OC5CC5)cn4)c(=O)c3)c2Cl)C(=O)N1] |
| | 3-(2-chloro-3-(1-((5-cyclopropoxypyridin-2-yl)methyl)-2-oxo-1,2- |
| | dihydropyridin-4-yl)phenyl)piperidine-2,6-dione |
| I-19 | 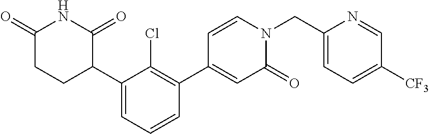 [SMILES: O=C1CCC(c2cccc(-c3ccn(Cc4ccc(C(F)(F)F)cn4)c(=O)c3)c2Cl)C(=O)N1] |
| | 3-(2-chloro-3-(2-oxo-1-((5-(trifluoromethyl)pyridin-2-yl)methyl)-1,2- |
| | dihydropyridin-4-yl)phenyl)piperidine-2,6-dione |
| I-20 | 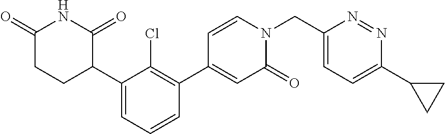 [SMILES: O=C1CCC(c2cccc(-c3ccn(Cc4ccc(C5CC5)nn4)c(=O)c3)c2Cl)C(=O)N1] |
| | 3-(2-chloro-3-(1-((6-cyclopropylpyridazin-3-yl)methyl)-2-oxo-1,2- |
| | dihydropyridin-4-yl)phenyl)piperidine-2,6-dione |
| I-21 | 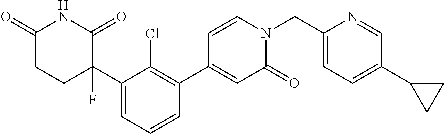 [SMILES: O=C1CCC(F)(c2cccc(-c3ccn(Cc4ccc(C5CC5)cn4)c(=O)c3)c2Cl)C(=O)N1] |
| | 3-(2-chloro-3-(1-((5-cyclopropylpyridin-2-yl)methyl)-2-oxo-1,2- |
| | dihydropyridin-4-yl)phenyl)-3-fluoropiperidine-2,6-dione |
| I-22 | 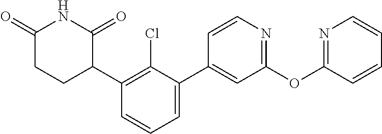 [SMILES: O=C1CCC(c2cccc(-c3ccnc(Oc4ccccn4)c3)c2Cl)C(=O)N1] |
| | 3-(2-chloro-3-(2-(pyridin-2-yloxy)pyridin-4-yl)phenyl)piperidine-2,6-dione |
| I-23 | 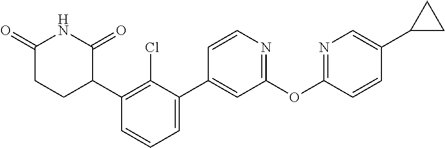 [SMILES: O=C1CCC(c2cccc(-c3ccnc(Oc4ccc(C5CC5)cn4)c3)c2Cl)C(=O)N1] |
| | 3-(2-chloro-3-(2-((5-cyclopropylpyridin-2-yl)oxy)pyridin-4- |
| | yl)phenyl)piperidine-2,6-dione |
| I-24 | 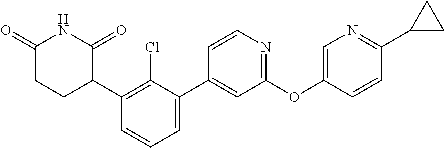 [SMILES: O=C1CCC(c2cccc(-c3ccnc(Oc4ccc(C5CC5)nc4)c3)c2Cl)C(=O)N1] |
| | 3-(2-chloro-3-(2-((6-cyclopropylpyridin-3-yl)oxy)pyridin-4- |
| | yl)phenyl)piperidine-2,6-dione |
| I-25. | 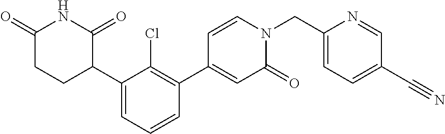 [SMILES: N#Cc1ccc(Cn2ccc(-c3cccc(C4CCC(=O)NC4=O)c3Cl)cc2=O)nc1] |
| | 6-((4-(2-chloro-3-(2,6-dioxopiperidin-3-yl)phenyl)-2-oxopyridin-1(2H)- |
| | yl)methyl)nicotinonitrile |
| I-26. | 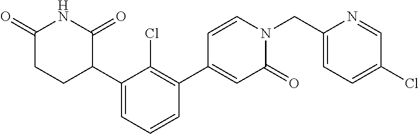 [SMILES: O=C1CCC(c2cccc(-c3ccn(Cc4ccc(Cl)cn4)c(=O)c3)c2Cl)C(=O)N1] |
| | 3-(2-chloro-3-(1-((5-chloropyridin-2-yl)methyl)-2-oxo-1,2-dihydropyridin- |
| | 4-yl)phenyl)piperidine-2,6-dione |
| I-27. | 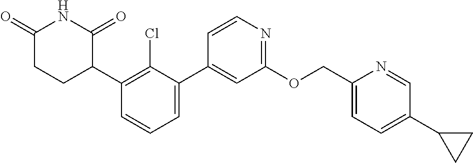 [SMILES: O=C1CCC(c2cccc(-c3ccnc(OCc4ccc(C5CC5)cn4)c3)c2Cl)C(=O)N1] |
| | 3-(2-chloro-3-(2-((5-cyclopropylpyridin-2-yl)methoxy)pyridin-4- |
| | yl)phenyl)piperidine-2,6-dione |
| I-28. | 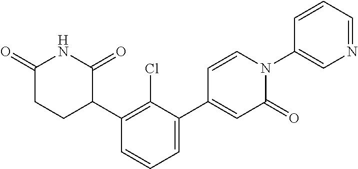 [SMILES: O=C1CCC(c2cccc(-c3ccn(-c4cccnc4)c(=O)c3)c2Cl)C(=O)N1] |
| | 3-(2-chloro-3-(2-oxo-2H-[1,3′-bipyridin]-4-yl)phenyl)piperidine-2,6-dione |
| I-29. | 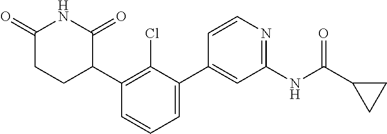 [SMILES: O=C1CCC(c2cccc(-c3ccnc(NC(=O)C4CC4)c3)c2Cl)C(=O)N1] |
| | N-(4-(2-chloro-3-(2,6-dioxopiperidin-3-yl)phenyl)pyridin-2- |
| | yl)cyclopropanecarboxamide |
| I-30. | 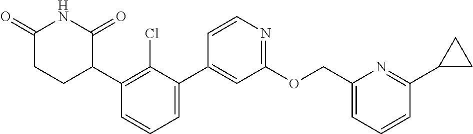 [SMILES: O=C1CCC(c2cccc(-c3ccnc(OCc4cccc(C5CC5)n4)c3)c2Cl)C(=O)N1] |
| | 3-(2-chloro-3-(2-((6-cyclopropylpyridin-2-yl)methoxy)pyridin-4- |
| | yl)phenyl)piperidine-2,6-dione |
| I-31. | 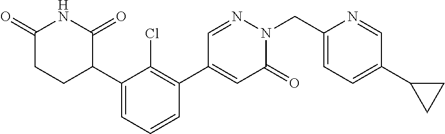 [SMILES: O=C1CCC(c2cccc(-c3cnn(Cc4ccc(C5CC5)cn4)c(=O)c3)c2Cl)C(=O)N1] |
| | 3-(2-chloro-3-(1-((5-cyclopropylpyridin-2-yl)methyl)-6-oxo-1,6- |
| | dihydropyridazin-4-yl)phenyl)piperidine-2,6-dione |
| I-32. | 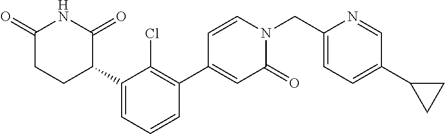 [SMILES: O=C1CC[C@@H](c2cccc(-c3ccn(Cc4ccc(C5CC5)cn4)c(=O)c3)c2Cl)C(=O)N1] |
| | (S)-3-(2-chloro-3-(1-((5-cyclopropylpyridin-2-yl)methyl)-2-oxo-1,2- |
| | dihydropyridin-4-yl)phenyl)piperidine-2,6-dione |
| I-33. | 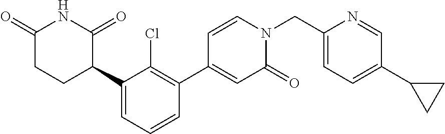 [SMILES: O=C1CC[C@H](c2cccc(-c3ccn(Cc4ccc(C5CC5)cn4)c(=O)c3)c2Cl)C(=O)N1] |
| | (R)-3-(2-chloro-3-(1-((5-cyclopropylpyridin-2-yl)methyl)-2-oxo-1,2- |
| | dihydropyridin-4-yl)phenyl)piperidine-2,6-dione |

The compounds of the present invention can effectively induce the degradation of VAV1, with a DC₅₀ preferably less than 100 nM. The compounds of the present invention can also effectively inhibit the activation of IL-2 induced by CD3/CD28, with an IC₅₀ preferably less than 100 nM. The compounds of the present invention can effectively alleviate disease progression in the mouse EAE model.

The invention also relates to a pharmaceutical composition, which comprises a compound of Formula I or a pharmaceutically acceptable salt, a stable isotope derivative, an isomer and a prodrug thereof, along with one or more pharmaceutically acceptable carriers or excipients. The pharmaceutical composition can induce the degradation of VAV1, affecting its biological function for treating or preventing diseases mediated by VAV1.

The present invention provides a method for treating or preventing diseases mediated by VAV1, which comprises administering to a patient in need a therapeutically effective amount of a compound of Formula I or a pharmaceutically acceptable salt, stable isotope derivative, isomer, prodrug and pharmaceutical composition thereof. The diseases include but are not limited to autoimmune related diseases (such as rheumatoid arthritis, systemic lupus erythematosus, psoriasis, multiple sclerosis, inflammatory bowel disease, neurodegenerative diseases, diabetes, etc.) and cancers (such as B or T-cell related hematologic tumors, solid tumors, etc.).

The invention also provides the use of a compound of Formula I or the pharmaceutically acceptable salt, stable isotope derivative, isomer, or pharmaceutical composition thereof for the treatment or prevention of diseases mediated by VAV1, where diseases mediated by VAV1 include but are not limited to autoimmune related diseases (such as rheumatoid arthritis, systemic lupus erythematosus, psoriasis, multiple sclerosis, inflammatory bowel disease, neurodegenerative diseases, diabetes, etc.) and cancer (such as B or T-cell related hematological tumors, solid tumors, etc.).

The invention further provides a compound of Formula I or the pharmaceutically acceptable salt, stable isotope derivative, isomer or pharmaceutical composition thereof for use in the preparation of a medication, where the medication is for the treatment or prevention of diseases mediated by VAV1, including but not limited to autoimmune related diseases (such as rheumatoid arthritis, systemic lupus erythematosus, psoriasis, multiple sclerosis, inflammatory bowel disease, neurodegenerative diseases, diabetes, etc.) and cancer (such as B or T-cell related hematological tumors, solid tumors, etc.).

According to the present invention, the pharmaceuticals can be in any dosage form, including but not limited to tablets, capsules, solutions, lyophilized preparations and injectables.

The pharmaceutical formulation of the present invention can be administered in a form of dosage units containing a predetermined amount of active ingredient. Depending on the disease being treated, the method of administration, as well as age, weight and condition of the patient, such a unit may contain 0.1 to 500 mg of a compound of the present invention. Furthermore, the pharmaceutical formulation can be prepared using methods well known in the pharmaceutical field, such as by formulating the active ingredient with one or more excipients or one or more adjuvants.

The pharmaceutical formulation of the present invention is suitable for administration by any appropriate method, such as oral (including buccal or sublingual), rectal, nasal, topical (including buccal, sublingual or transdermal) or parenteral (including subcutaneous, intramuscular, intravenous or intradermal) administration.

The invention also provides a method for preparing the compounds. The compounds can be synthesized using synthetic methods known to those skilled in the art. The products obtained from each step of the reaction are isolated by separation techniques known in the field. The starting materials and chemical reagents used for the synthesis can be conventionally made according to the literature which can be searched from SciFinder or purchased.

The heterocyclic compounds shown in Formula I of the present invention can be synthesized by Suzuki coupling reaction from suitable halide A and borate B, or borate compound A′ and halide B′. I can be further derivatized to give other target compounds according to conventional chemical reactions.

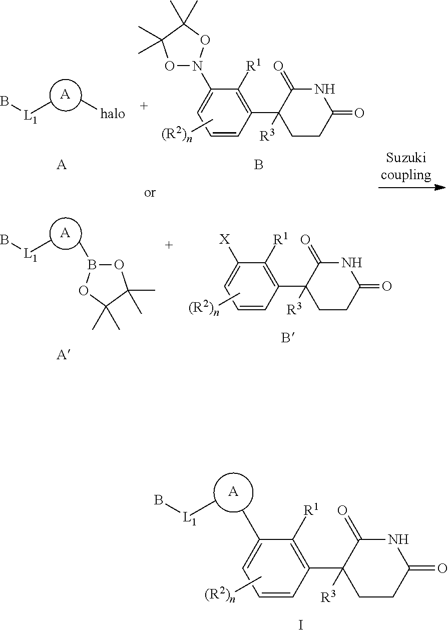

Halide B′ and borate B can be synthesized according to the following route: a) B-1 is brominated by NBS, then substituted with cyano to give B-2; b) B-2 can undergo further substitution reactions to generate B-3; c) B-3 undergoes addition with tert-butyl acrylate in the presence of a base to give B-4; d) B-4 undergoes cyclization under acid catalysis to give B′; e) B′ is converted into borate B under palladium catalysis.

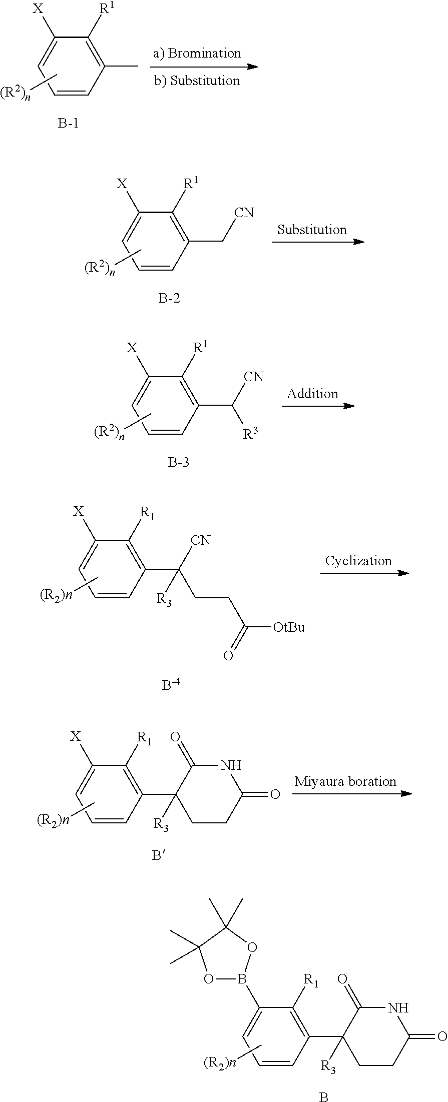

The following examples further illustrate the present invention. These examples are intended solely to illustrate the invention and should not be considered as limiting the scope of the invention.

### EXAMPLES

The starting materials of the present invention were synthesized according to methods known in the art, or purchased from chemical companies such as ABCR GmbH & Co. KG, Acros Organics, Aldrich Chemical Company, Accela ChemBio Inc., Beijing Coupling, Bide Pharmatech, Zesheng Technology, etc.

The structures of the compounds of the present invention were determined by nuclear magnetic resonance (NMR) and/or mass spectrometry (MS). The NMR measurement used a Bruker ASCEND-400 NMR spectrometer, with a solvent, such as deuterated dimethyl sulfoxide (DMSO-d₆), deuterated chloroform (CDCl₃), deuterated methanol (CD₃OD), and tetramethylsilane (TMS) as the internal standard. Chemical shift was given in a unit of 10⁻⁶ (ppm). MS measurement used an Agilent SQD (ESI) mass spectrometer (Agilent 6120).

HPLC used an Agilent 1260 DAD high-performance liquid chromatography system (Poroshell 120 EC-C18, 50×3.0 mm, 2.7 μm column) or a Waters Arc high-performance liquid chromatography system (Sunfire C18, 150×4.6 mm, 5 μm column).

Unless otherwise stated, the reaction was run at room temperature (20° C.-30° C.).

Unless otherwise stated, the reaction was carried out under an argon or nitrogen atmosphere. An argon or nitrogen atmosphere refers to a reaction flask connected to an approximately 1-L volume balloon filled with argon or nitrogen.

A hydrogen atmosphere refers to the reaction flask being evacuated and then filled with hydrogen gas (repeated 3 times), followed by connecting a hydrogen balloon with a volume of approximately 1 L.

The microwave reaction used a CEM Discover-SP type microwave reactor.

The reaction was monitored using an Agilent's liquid chromatography-mass spectrometry (LC-MS) system (1260/6120) and thin-layer chromatography (TLC) with a thickness of the silica gel plate being 0.15~0.2 mm (Qingdao Haiyang GF254).

The compound purification was carried out by column chromatography or thin-layer chromatography, where column chromatography used 200~300 mesh silica gel from Qingdao Haiyang and thin-layer chromatography used GF254 silica gel plates with a thickness of 0.4~0.5 mm from Qingdao Haiyang.

Column chromatography or thin-layer chromatography eluent solvent systems typically included a) dichloromethane and methanol, b) ethyl acetate and hexane, or as shown in examples. The ratio of solvents was adjusted according to the polarity of the compound and further adjusted by addition of a small amount of triethylamine or other acidic or basic reagents.

The purification of the compounds also used a mass spectrometry-guided automated preparation system from Waters (mass spectrometer: SQD2). Depending on the polarity of the compound, an appropriate acetonitrile/water gradient (containing 0.1% trifluoroacetic acid or formic acid, or 0.05% ammonia) was used to elute the reverse-phase high-pressure column (XBridge-C18, 19×150 mm, 5 m) at a flow rate of 20 mL/min.

The abbreviation DMSO refers to dimethyl sulfoxide.

The abbreviation DMF refers to N,N-dimethylformamide.

The abbreviation THF refers to tetrahydrofuran.

The abbreviation TBAF refers to tetrabutylammonium fluoride.

The abbreviation Pd₂(dba)₃ refers to tris(dibenzylideneacetone)dipalladium.

The abbreviation Pd(dppf)Cl₂ refers to [1,1′-bis(diphenylphosphino)ferrocene]dichloropalladium.

The abbreviation NBS refers to N-bromosuccinimide.

The abbreviation AIBN refers to azodiisobutyronitrile.

The abbreviation TMSCN refers to trimethylsilyl cyanide.

The abbreviation DMA refers to N,N-dimethylacetamide.

The abbreviation DIPEA refers to N,N-diisopropylethylamine.

The abbreviation LiAlH₄ refers to lithium aluminum hydride.

The abbreviation NaHMDS refers to sodium bis(trimethylsilyl)amide.

The abbreviation NFSI refers to N-fluoro-N-phenylsulfonyl benzenesulfonamide.

The abbreviation TBSCl refers to tert-butyldimethylsilyl chloride.

The abbreviation Pd(PPh₃)₄ refers to tetrakis(triphenylphosphine)palladium.

The abbreviation prep-HPLC refers to preparative reversed-phase high-performance liquid chromatography.

### Example 1. Synthesis of Intermediates

### Intermediate A: 3-(3-bromo-2-chlorophenyl)piperidine-2,6-dione

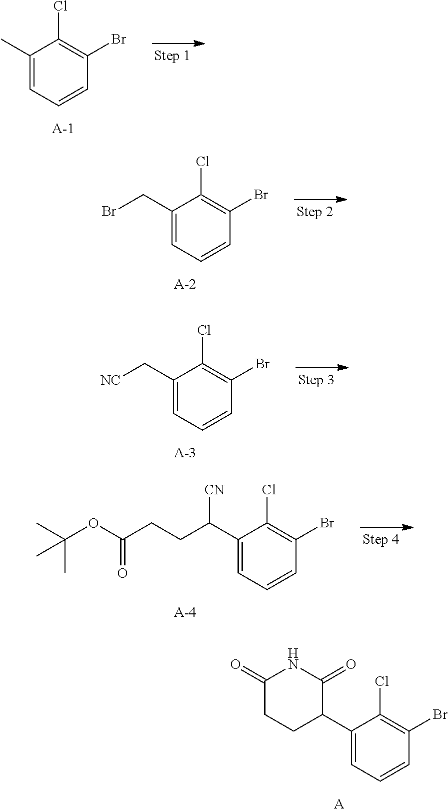

### Step 1

### 1-Bromo-3-(bromomethyl)-2-chlorobenzene (A-2)

To a mixture of 1-bromo-2-chloro-3-methylbenzene A-1 (2.9 g, 14 mmol), NBS (2.6 g, 15 mmol) and carbon tetrachloride (28 mL) was added AIBN (0.23 g, 1.4 mmol). The reaction mixture was stirred under reflux for 30 minutes. The mixture was filtered, and the filtrate was concentrated under reduced pressure. The residue was purified by silica gel column chromatography (petroleum ether/ethyl acetate=100/0 to 0/100 v/v) to give the target product A-2 (2.4 g, 60%).

### Step 2

### 2-(3-Bromo-2-chlorophenyl)acetonitrile (A-3)

To a mixture of A-2 (2.4 g, 8.4 mmol) and dichloromethane (25 mL) at 0° C. were added TMSCN (1.6 mL, 12.6 mmol) and a THF solution of TBAF (1.0 M, 13 mL, 13 mmol). The reaction mixture was stirred at room temperature for 1.5 hours. The mixture was quenched with water and extracted with dichloromethane. The organic phase was concentrated under reduced pressure, and the residue was purified by silica gel column chromatography (petroleum ether/ethyl acetate=100/0 to 0/100 v/v) to give the target product A-3 (1.7 g, 86%).

### Step 3

### tert-Butyl 4-(3-bromo-2-chlorophenyl)-4-cyanobutanoate (A-4)

To a mixture of A-3 (1.66 g, 7.25 mmol), tert-butyl acrylate (1.1 mL, 7.25 mmol), and THF (22 mL) at 0° C. was added sodium methoxide (78 mg, 1.45 mmol). The reaction mixture was stirred at room temperature for 2 hours, quenched with water and extracted with dichloromethane. The organic phase was concentrated under reduced pressure, and the residue was purified by silica gel column chromatography (petroleum ether/ethyl acetate=100/0 to 0/100 v/v) to give the target product A-4 (2.2 g, 96%).

MS m/z (ESI): 358.1 [M+H⁺]

### Step 4

### 3-(3-Bromo-2-chlorophenyl)piperidine-2,6-dione (A)

To a mixture of A-4 (2.5 g, 7.0 mmol) and acetic acid (20 mL) was added concentrated sulfuric acid (2 mL), and the mixture was stirred at 90° C. for 3 hours. After cooling to room temperature, the mixture was added with ice water. The resulting mixture was filtered, and the filter cake was washed with water and dried to give the target product A (1.7 g, 82%).

MS m/z (ESI): 301.9 [M+H⁺]

### Intermediate B: 3-(2-chloro-3-(4,4,5,5-tetramethyl-1,3,2-dioxaborolan-2-yl)phenyl)piperidine-2,6-dione

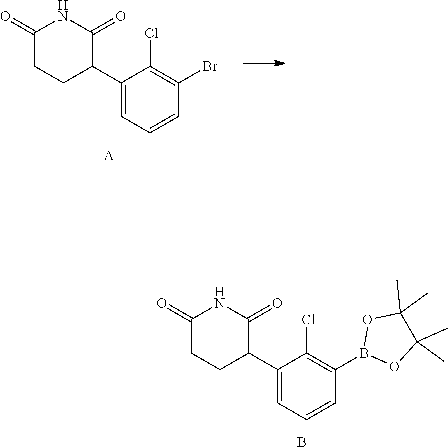

To a solution of A (500 mg, 1.7 mmol) in 1,4-dioxane (10 mL) were added 4,4,4′,4′,5,5,5′,5′-octamethyl-2,2′-bi(1,3,2-dioxaborolane) (633 mg, 2.5 mmol), Pd(dppf)Cl₂ (122 mg, 0.17 mmol) and potassium acetate (326 mg, 3.3 mmol). The mixture was stirred at 90° C. for 2 hours. After cooled to room temperature, the mixture was concentrated under reduced pressure. The residue was purified by prep-HPLC to give the target product B (290 mg, 50%).

MS m/z (ESI): 350.2 [M+H⁺]

### Example 2

### 3-(2-Chloro-3-(2-oxo-1-phenyl-1,2-dihydropyridin-4-yl)phenyl)piperidine-2,6-dione (Compound I-1)

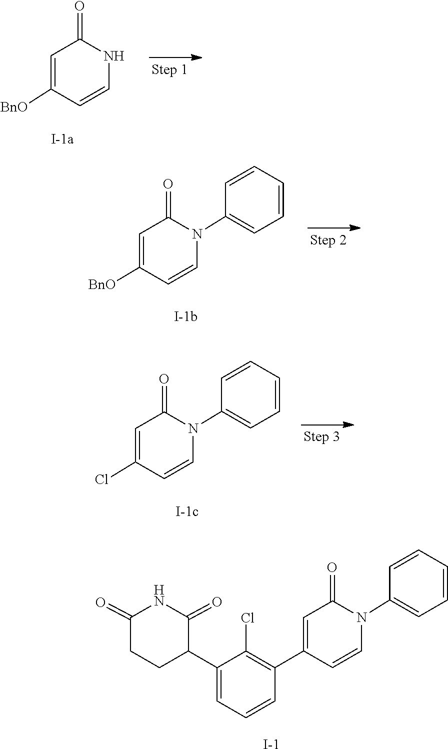 [SMILES: O=c1cc(OCc2ccccc2)cc[nH]1 ; O=c1cc(OCc2ccccc2)ccn1-c1ccccc1 ; O=c1cc(Cl)ccn1-c1ccccc1 ; O=C1CCC(c2cccc(-c3ccn(-c4ccccc4)c(=O)c3)c2Cl)C(=O)N1 ; [I-]]

### Step 1

### 4-(Benzyloxy)-1-phenylpyridin-2(1H)-one (I-1b)

To a mixture of phenylboronic acid (1 g, 8.2 mmol), 4-(benzyloxy)pyridin-2(1H)-one I-1a (1.98 g, 9.8 mmol) and 1,2-dichloroethane (30 mL) were added copper acetate (1.4 g, 8.2 mmol) and triethylamine (3.4 mL, 24.6 mmol). The mixture was stirred at room temperature in air for 3 hours and filtered. The filtrate was concentrated under reduced pressure and the residue was purified by silica gel column chromatography (petroleum ether/ethyl acetate=2/1 to 1/1 v/v) to give the target product I-1b (1.2 g, 55%).

MS m/z (ESI): 278.1 [M+H⁺]

### Step 2

### 4-Chloro-1-phenylpyridin-2(1H)-one (I-1c)

To a solution of I-1b (100 mg, 0.36 mmol) in 1,2-dichloroethane (30 mL) was added phosphoryl tribromide (414 mg, 1.4 mmol), and the mixture was stirred at 150° C. in a microwave reactor for 30 minutes. After cooling to room temperature, the mixture was quenched with saturated sodium bicarbonate solution and extracted with dichloromethane. The organic phase was dried over anhydrous sodium sulfate, filtered, and concentrated under reduced pressure. The residue was purified by silica gel column chromatography (dichloromethane/methanol=50/1 to 15/1 v/v) to give the target product I-1c (60 mg, 80%).

MS m/z (ESI): 206.0 [M+H⁺]

### Step 3

### 3-(2-Chloro-3-(2-oxo-1-phenyl-1,2-dihydropyridin-4-yl)phenyl)piperidine-2,6-dione (I-1)

To a mixture of I-1c (26 mg, 0.13 mmol) and anhydrous 1,4-dioxane (5 mL) were added 4,4,4′,4′,5,5,5′,5′-octamethyl-2,2′-bi(1,3,2-dioxaborolane) (64 mg, 0.25 mmol), Pd₂(dba)₃ (12 mg, 0.013 mmol), tricyclohexylphosphonium tetrafluoroborate (9 mg, 0.025 mmol) and potassium acetate (37 mg, 0.38 mmol). The mixture was stirred at 100° C. for 3 hours and then concentrated under reduced pressure. The residue was dissolved in DMF (5 mL), added with Pd(dppf)Cl₂ (9 mg, 0.013 mmol), A (38 mg, 0.13 mmol) and potassium phosphate (81 mg, 0.38 mmol). The reaction mixture was stirred at 100° C. for additional 1 hour and concentrated under reduced pressure. The residue was purified by prep-HPLC to give the target product I-1 (13 mg, 26%).

MS m/z (ESI): 393.1 [M+H⁺]

¹H NMR (400 MHz, DMSO-d₆) δ 10.94 (s, 1H), 7.73 (d, J=7.1 Hz, 1H), 7.58-7.38 (m, 8H), 6.49 (d, J=1.6 Hz, 1H), 6.38 (dd, J=7.1, 1.9 Hz, 1H), 4.37 (dd, J=12.3, 5.0 Hz, 1H), 2.87-2.74 (m, 1H), 2.61-2.55 (m, 1H), 2.41-2.28 (m, 1H), 2.10-1.93 (m, 2H).

### Example 3

### 3-(2-Chloro-3-(1-((1-methyl-1H-pyrazol-3-yl)methyl)-2-oxo-1,2-dihydropyridin-4-yl)phenyl)piperidine-2,6-dione (Compound I-2)

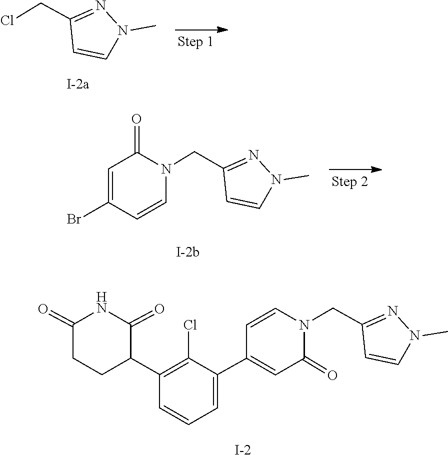

### Step 1

### 4-Bromo-1-((1-methyl-1H-pyrazol-3-yl)methyl)pyridin-2(1H)-one (I-2b)

To a solution of 3-(chloromethyl)-1-methyl-1H-pyrazole I-2a (1.0 g, 7.6 mmol) and 4-bromopyridin-2(1H)-one (1.3 g, 7.6 mmol) in DMF (20 mL) was added cesium carbonate (5.0 g, 15 mmol), and the mixture was stirred at room temperature overnight. The mixture was filtered, and the filter cake was washed with ethyl acetate. The filtrate was concentrated under reduced pressure. The residue was purified by silica gel column chromatography (dichloromethane/methanol=97/3 v/v) to give the target product I-2b (1.9 g, 95%).

MS m/z (ESI): 268.1 [M+H⁺]

### Step 2

### 3-(2-chloro-3-(1-((1-methyl-1H-pyrazol-3-yl)methyl)-2-oxo-1,2-dihydropyridin-4-yl)phenyl)piperidine-2,6-dione (I-2)

To a solution of I-2b (200 mg, 0.75 mmol) in 1,4-dioxane (4 mL) were added 4,4,4′,4′,5,5,5′,5′-octamethyl-2,2′-bi(1,3,2-dioxaborolane) (285 mg, 1.1 mmol), Pd(dppf)Cl₂ (55 mg, 0.075 mmol) and potassium acetate (147 mg, 1.5 mmol), and the mixture was stirred at 90° C. for 2 hours. After cooling to room temperature, the mixture was concentrated under reduced pressure. The residue was dissolved in DMF (3 mL), added with A (338 mg, 1.1 mmol), Pd(dppf)Cl₂ (55 mg, 0.075 mmol) and potassium phosphate (318 mg, 1.5 mmol), and the reaction mixture was stirred at 100° C. overnight. The mixture was filtered, and the filter cake was washed with ethyl acetate. The filtrate was concentrated under reduced pressure. The residue was purified by prep-HPLC to give the target product I-2 (141 mg, 46%).

MS m/z (ESI): 411.1 [M+H⁺]

¹H NMR (400 MHz, DMSO-d₆) δ 10.92 (s, 1H), 7.72 (d, J=7.0 Hz, 1H), 7.63 (d, J=2.1 Hz, 1H), 7.43-7.38 (m, 2H), 7.35-7.31 (m, 1H), 6.37 (d, J=1.9 Hz, 1H), 6.27 (dd, J=7.0, 2.0 Hz, 1H), 6.20 (d, J=2.2 Hz, 1H), 5.06 (s, 2H), 4.34 (dd, J=12.2, 5.0 Hz, 1H), 3.81 (s, 3H), 2.84-2.74 (m, 1H), 2.58-2.53 (m, 1H), 2.38-2.25 (m, 1H), 2.07-1.98 (m, 1H).

### Example 4

### 3-(2-Chloro-3-(2-oxo-1-((tetrahydro-2H-pyran-4-yl)methyl)-1,2-dihydropyridin-4-yl)phenyl)piperidine-2,6-dione (Compound I-3)

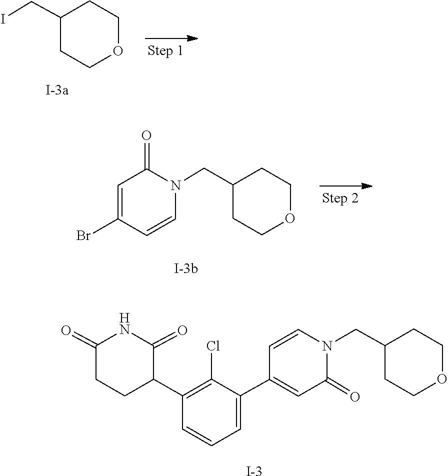

### Step 1

### 4-Bromo-1-((tetrahydro-2H-pyran-4-yl)methyl)pyridin-2(1H)-one (I-3b)

I-3b was synthesized according to the procedure of Step one for Example 3, but I-3a was used instead of I-2a.

MS m/z (ESI): 272.1 [M+H⁺]

### Step 2

### 3-(2-chloro-3-(2-oxo-1-((tetrahydro-2H-pyran-4-yl)methyl)-1,2-dihydropyridin-4-yl)phenyl)piperidine-2,6-dione (I-3)

To a solution of I-3b (50 mg, 0.19 mmol) in DMF (3 mL) were added B (64 mg, 0.19 mmol), Pd(dppf)Cl₂ (14 mg, 0.019 mmol) and potassium phosphate (78 mg, 0.37 mmol). The reaction mixture was stirred at 100° C. overnight and filtered. The filter cake was washed with ethyl acetate. The filtrate was concentrated under reduced pressure. The residue was purified by prep-HPLC to give the target product I-3 (2.6 mg, 3%).

MS m/z (ESI): 415.1 [M+H⁺]

¹H NMR (400 MHz, DMSO-d₆) δ 10.92 (s, 1H), 7.70 (d, J=6.9 Hz, 1H), 7.43-7.38 (m, 2H), 7.36-7.31 (m, 1H), 6.36 (d, J=1.8 Hz, 1H), 6.25 (dd, J=6.9, 2.0 Hz, 1H), 4.34 (dd, J=12.2, 5.1 Hz, 1H), 3.89-3.78 (m, 4H), 3.28-3.20 (m, 2H), 2.84-2.74 (m, 1H), 2.58-2.53 (m, 1H), 2.38-2.26 (m, 1H), 2.12-1.95 (m, 2H), 1.51-1.43 (m, 2H), 1.36-1.25 (m, 2H).

Compound I-4 was synthesized according to the procedures for Example 4, but a different reagent was used instead of I-3a.

| Compound | | Compound replacing | MS m/z |
|---|---|---|---|
| Number | Compound Structure | I-3a | (ESI) |
| I-4 | 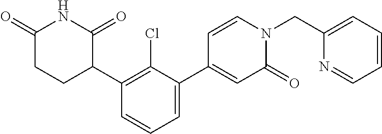 [SMILES: O=C1CCC(c2cccc(-c3ccn(Cc4ccccn4)c(=O)c3)c2Cl)C(=O)N1] | 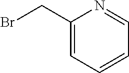 | 408.1 [M + H⁺] |

The NMR data for compound I-4 are as follows:

| Compound | ¹H NMR |
|---|---|
| 3-(2-chloro-3-(2-oxo-1- | ¹H NMR (400 MHz, DMSO-d₆) δ 10.92 (s, 1H), 8.53 (s, |
| (pyridin-2-ylmethyl)- | 1H), 7.93-7.74 (m, 2H), 7.46-7.25 (m, 5H), 6.39 (s, 1H), |
| 1,2-dihydropyridin-4- | 6.33 (d, J = 6.7 Hz, 1H), 5.23 (s, 2H), 4.41-4.28 (m, 1H), |
| yl)phenyl)piperidine- | 2.86-2.72 (m 1H), 2.62-2.55 (m, 1H), 2.42-2.28 (m, |
| 2,6-dione (I-4) | 1H), 2.11-1.96 (m, 1H). |

### Example 5

### 3-(2-Chloro-3-(1-((6-cyclopropylpyridin-2-yl)methyl)-2-oxo-1,2-dihydropyridin-4-yl)phenyl)piperidine-2,6-dione (Compound I-5)

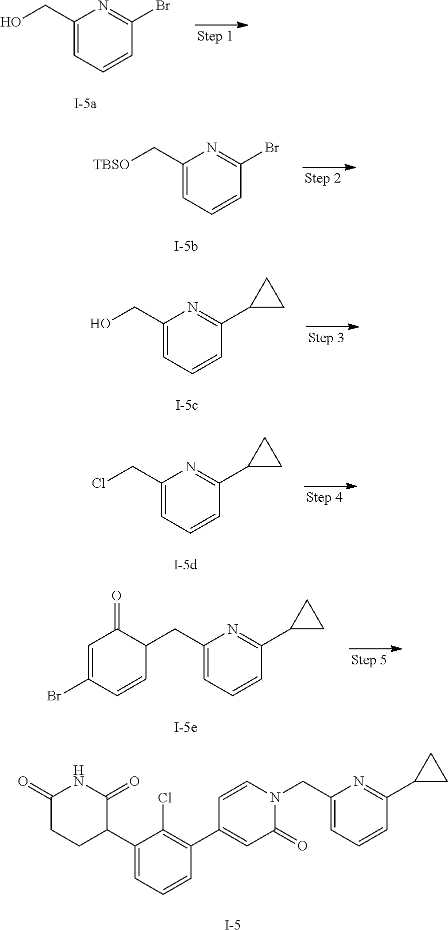

### Step 1

### 2-Bromo-6-(((tert-butyldimethylsilyl)oxy)methyl)pyridine (I-5b)

A mixture of I-5a (1.87 g, 10.0 mmol), imidazole (817 mg, 12.0 mmol), TBSCl (1.81 g, 12.0 mmol) and dichloromethane (30 mL) was stirred at room temperature overnight. It was concentrated under reduced pressure and the residue was purified by silica gel column chromatography to give the target product I-5b (2.4 g, 80%).

MS m/z (ESI): 302.1 [M+H⁺]

### Step 2

### (6-Cyclopropylpyridin-2-yl)methanol (I-5c)

A mixture of I-5b (2.4 g, 8.0 mmol), cyclopropylboronic acid (1.37 g, 16.0 mmol), potassium phosphate (5.1 g, 24.0 mmol), Pd(dppf)Cl₂ (590 mg, 0.8 mmol), 1,4-dioxane (48 mL) and water (16 mL) was stirred at 95° C. overnight. After cooling to room temperature, the reaction mixture was added with diluted hydrochloric acid aqueous solution (1 N) and extracted with ethyl acetate. The organic phase was concentrated under reduced pressure and the residue was purified by silica gel column chromatography to give the target product I-5c (780 mg, 65%).

MS m/z (ESI): 150.1 [M+H⁺]

### Step 3

### 2-(Chloromethyl)-6-cyclopropylpyridine (I-5d)

A mixture of I-5c (740 mg, 5.0 mmol) and thionyl chloride (7 mL) was stirred at room temperature for 1 hour. It was concentrated under reduced pressure to yield the target product I-5d (crude, 860 mg). The product was used directly in the next step without further purification.

MS m/z (ESI): 168.1 [M+H⁺]

### Steps 4-5

### 3-(2-Chloro-3-(1-((6-cyclopropylpyridin-2-yl)methyl)-2-oxo-1,2-dihydropyridin-4-yl)phenyl)piperidine-2,6-dione (I-5)

Compound I-5 was synthesized according to the procedures of Steps 1-2 for Example 3, but I-5d was used instead of I-2a.

MS m/z (ESI): 448.1 [M+H⁺]

¹H NMR (400 MHz, DMSO-d₆) δ 10.92 (s, 1H), 7.78 (d, J=7.0 Hz, 1H), 7.62 (t, J=7.7 Hz, 1H), 7.45-7.39 (m, 2H), 7.35 (dd, J=5.5, 3.8 Hz, 1H), 7.19 (d, J=7.5 Hz, 1H), 6.99 (d, J=7.4 Hz, 1H), 6.36 (d, J=1.8 Hz, 1H), 6.30 (dd, J=7.0, 2.0 Hz, 1H), 5.15 (s, 2H), 4.35 (dd, J=12.3, 5.0 Hz, 1H), 2.86-2.75 (m, 1H), 2.58-2.53 (m, 1H), 2.38-2.27 (m, 1H), 2.08-1.99 (m, 2H), 0.95-0.77 (m, 4H).

### Example 6

### 3-(2-Chloro-3-(1-((5-cyclopropylpyridin-2-yl)methyl)-2-oxo-1,2-dihydropyridin-4-yl)phenyl)piperidine-2,6-dione (Compound I-6)

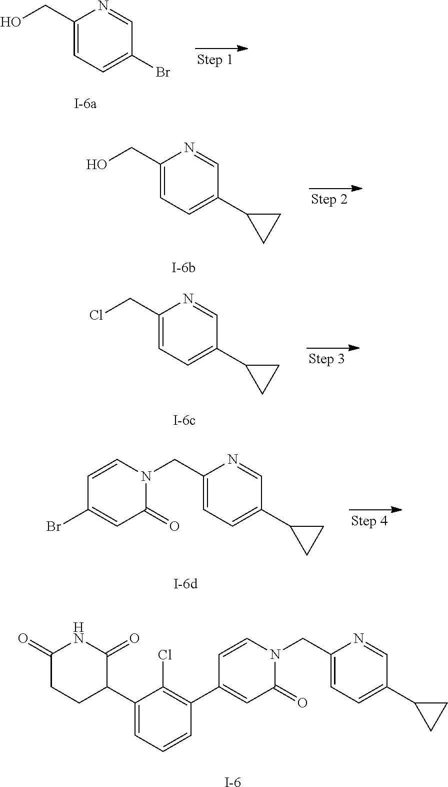

### Step 1

### (5-Cyclopropylpyridin-2-yl)methanol (I-6b)

A mixture of I-6a (2.0 g, 11 mmol), cyclopropylboronic acid (1.8 g, 21 mmol), potassium phosphate (6.7 g, 32 mmol), Pd(dppf)Cl₂ (774 mg, 1.1 mmol), 1,4-dioxane (20 mL) and water (6 mL) was stirred at 95° C. overnight. After cooling to room temperature, the reaction mixture was added with water and extracted with ethyl acetate. The organic phase was concentrated under reduced pressure and the residue was purified by silica gel column chromatography (petroleum ether/ethyl acetate=15/85 v/v) to give the target product I-6b (549 mg, 35%).

MS m/z (ESI): 150.1 [M+H⁺]

### Step 2

### 2-(Chloromethyl)-5-cyclopropylpyridine (I-6c)

A mixture of I-6b (549 mg, 3.7 mmol) and thionyl chloride (5 mL) was stirred at 65° C. for 1 hour. The reaction mixture was concentrated under reduced pressure to yield the target product I-6c (crude, 610 mg). The product was used directly in the next step without further purification.

MS m/z (ESI): 168.1 [M+H⁺]

### Step 3

### 4-Bromo-1-((5-cyclopropylpyridin-2-yl)methyl)pyridin-2(1H)-one (I-6d)

To a solution of 3-(chloromethyl)-1-methyl-1H-pyrazole I-6c (610 mg, crude) and 4-bromopyridin-2(1H)-one (622 mg, 3.6 mmol) in DMF (6 mL) was added cesium carbonate (3.5 g, 11 mmol), and the mixture was stirred at room temperature overnight. The mixture was added with water and extracted with ethyl acetate. The organic phase was concentrated under reduced pressure and the residue was purified by silica gel column chromatography (petroleum ether/ethyl acetate=54/46 v/v) to give the target product I-6d (360 mg, 32% yield for two steps).

MS m/z (ESI): 305.1 [M+H⁺]

### Step 4

### 3-(2-Chloro-3-(1-((5-cyclopropylpyridin-2-yl)methyl)-2-oxo-1,2-dihydropyridin-4-yl)phenyl)piperidine-2,6-dione (I-6)

A mixture of I-6d (110 mg, 0.36 mmol), 4,4,4′,4′,5,5,5′,5′-octamethyl-2,2′-bi(1,3,2-dioxaborolane) (137 mg, 0.54 mmol), Pd(dppf)Cl₂ (26 mg, 0.036 mmol) and potassium acetate (176 mg, 1.1 mmol) in 1,4-dioxane (3 mL) was stirred at 90° C. for 2 hours. After cooling to room temperature, the mixture was added with A (218 mg, 0.72 mmol), Pd(dppf)Cl₂ (26 mg, 0.036 mmol) and potassium phosphate (229 mg, 1.1 mmol), and then stirred at 95° C. overnight. The mixture was added with water and extracted with ethyl acetate. The organic phase was concentrated under reduced pressure. The residue was purified by silica gel column chromatography (petroleum ether/ethyl acetate=20/80 v/v), and further purified by prep-HPLC to give the target product I-6 (70 mg, 43%).

MS m/z (ESI): 448.1 [M+H⁺]

¹H NMR (400 MHz, DMSO-d₆) δ 10.92 (s, 1H), 8.36 (d, J=2.1 Hz, 1H), 7.83 (d, J=7.0 Hz, 1H), 7.46-7.39 (m, 3H), 7.38-7.32 (m, 1H), 7.20 (d, J=8.1 Hz, 1H), 6.37 (d, J=1.9 Hz, 1H), 6.31 (dd, J=7.0, 2.0 Hz, 1H), 5.16 (s, 2H), 4.35 (dd, J=12.3, 5.0 Hz, 1H), 2.87-2.73 (m, 1H), 2.59-2.53 (m, 1H), 2.38-2.27 (m, 1H), 2.08-2.00 (m, 1H), 1.98-1.89 (m, 1H), 1.04-0.95 (m, 2H), 0.77-0.68 (m, 2H).

### Example 7

### 3-(2-Chloro-3-(2-oxo-1-(tetrahydro-2H-pyran-4-yl)-1,2-dihydropyridin-4-yl)phenyl)piperidine-2,6-dione (Compound I-7)

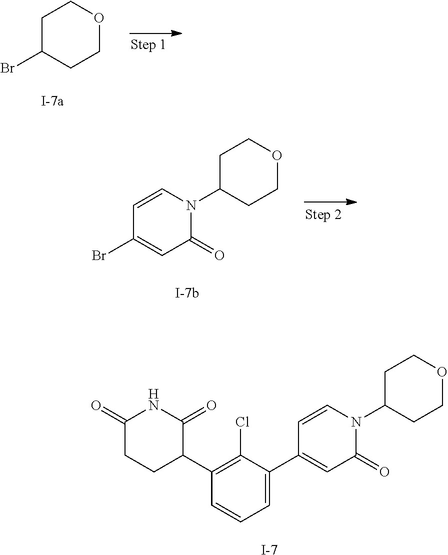

### Step 1

### 4-Bromo-1-(tetrahydro-2H-pyran-4-yl)pyridin-2(1H)-one (I-7b)

To a solution of 4-bromotetrahydro-2H-pyran I-7a (948 mg, 5.7 mmol) and 4-bromopyridin-2(1H)-one (1 g, 5.7 mmol) in DMF (20 mL) was added cesium carbonate (3.55 g, 10.9 mmol), and the reaction mixture was stirred at 100° C. overnight. The mixture was filtered, the filter cake was washed with ethyl acetate, and the filtrate was concentrated under reduced pressure. The residue was purified by silica gel column chromatography (dichloromethane/methanol=97/3) to give the target product I-7b (210 mg, 14%).

MS m/z (ESI): 258.0 [M+H⁺]

### Step 2

### 3-(2-Chloro-3-(2-oxo-1-(tetrahydro-2H-pyran-4-yl)-1,2-dihydropyridin-4-yl)phenyl)piperidine-2,6-dione (I-7)

Compound 1-7 was synthesized according to the procedure of Step 2 for Example 4, but I-7b was used instead of I-3b.

MS m/z (ESI): 401.1 [M+H⁺]

¹H NMR (400 MHz, DMSO-d₆) δ 10.92 (s, 1H), 7.82 (d, J=7.2 Hz, 1H), 7.44-7.38 (m, 2H), 7.35 (t, J=4.7 Hz, 1H), 6.38 (d, J=1.9 Hz, 1H), 6.33-6.28 (m, 1H), 5.01-4.88 (m, 1H), 4.34 (dd, J=12.3, 5.0 Hz, 1H), 4.00 (dd, J=11.1, 4.0 Hz, 2H), 3.49 (dd, J=11.7, 10.2 Hz, 2H), 2.84-2.75 (m, 1H), 2.58-2.53 (m, 1H), 2.38-2.25 (m, 1H), 2.08-2.00 (m, 1H), 1.98-1.86 (m, 2H), 1.78-1.69 (m, 2H).

Compound I-26 was synthesized following the procedures for Example 7, but a different reagent was used instead of I-7a.

| Compound | | Compound replacing | MS m/z |
|---|---|---|---|
| Number | Compound Structure | I-7a | (ESI) |
| I-26 | 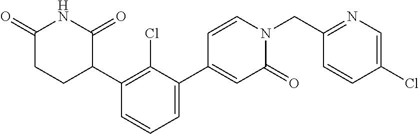 [SMILES: O=C1CCC(c2cccc(-c3ccn(Cc4ccc(Cl)cn4)c(=O)c3)c2Cl)C(=O)N1] | 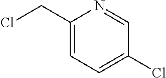 | 442.0 [M + H⁺] |

The NMR data for compound I-26 are as follows:

| Compound | ¹H NMR |
|---|---|
| 3-(2-chloro-3-(1-((5- | ¹H NMR (400 MHz, DMSO-d₆) δ 10.92 (s, 1H), 8.59 (d, J = 2.4 Hz, 1H), |
| chloropyridin-2-yl)methyl)- | 7.94 (dd, J = 8.4, 2.5 Hz, 1H), 7.86 (d, J = 7.0 Hz, 1H), 7.47-7.32 (m, |
| 2-oxo-1,2-dihydropyridin-4- | 4H), 6.39 (d, J = 1.8 Hz, 1H), 6.34 (dd, J = 7.0, 2.0 Hz, 1H), 5.23 (s, 2H), |
| yl)phenyl)piperidine-2,6- | 4.35 (dd, J = 12.3, 5.0 Hz, 1H), 2.85-2.74 (m, 1H), 2.60-2.53 (m, 1H), |
| dione (I-26) | 2.40-2.26 (m, 1H), 2.08-1.98 (m, 1H). |

### Example 8

### 3-(2-chloro-3-(2-((tetrahydro-2H-pyran-4-yl)oxy)pyridin-4-yl)phenyl)piperidine-2,6-dione (Compound I-8)

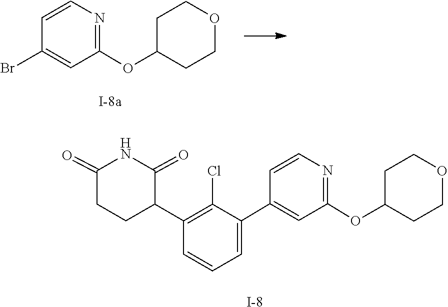

Compound I-8 was synthesized according to the procedure of Step 2 for Example 4, but I-8a was used instead of I-3b.

MS m/z (ESI): 401.1[M+H⁺]

¹H NMR (400 MHz, DMSO-d₆) δ 10.92 (s, 1H), 8.22 (d, J=5.2 Hz, 1H), 7.47-7.38 (m, 2H), 7.35 (dd, J=5.3, 4.0 Hz, 1H), 7.01 (dd, J=5.3, 1.4 Hz, 1H), 6.79 (s, 1H), 5.25-5.09 (m, 1H), 4.35 (dd, J=12.2, 5.0 Hz, 1H), 3.92-3.83 (m, 2H), 3.56-3.44 (m, 2H), 2.86-2.73 (m, 1H), 2.58-2.53 (m, 1H), 2.39-2.26 (m, 1H), 2.10-1.98 (m, 3H), 1.73-1.57 (m, 2H).

### Example 9

### 3-(2-Chloro-3-(2-oxo-2H-[1,2′-bipyridin]-4-yl)phenyl)piperidine-2,6-dione (Compound I-9)

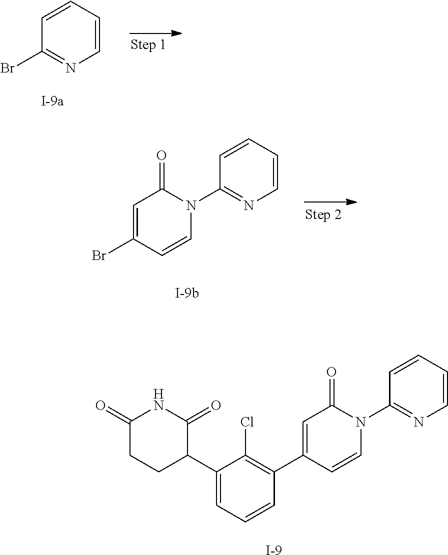

### Step 1

### 4-Bromo-2H-[1,2′-bipyridin]-2-one (I-9b)

To a solution of I-9a (500 mg, 2.8 mmol) and 4-bromopyridin-2(1H)-one (908 mg, 5.7 mmol) in toluene (12 mL) were added N,N′-diethylenediamine (25 mg, 0.3 mmol), copper(I) iodide (27 mg, 0.1 mmol) and potassium phosphate (1.2 g, 5.7 mmol), and the mixture was stirred at 120° C. overnight. The reaction mixture was concentrated under reduced pressure and the residue was purified by silica gel column chromatography (petroleum ether/ethyl acetate=100/0 to 30/70 v/v) to give the target product I-9b (0.4 g, 55%).

MS m/z (ESI): 251.0 [M+H⁺]

### Step 2

### 3-(2-Chloro-3-(2-oxo-2H-[1,2′-bipyridin]-4-yl)phenyl)piperidine-2,6-dione (I-9)

To a solution of I-9b (200 mg, 0.8 mmol) in 1,4-dioxane (10 mL) were added 4,4,4′,4′,5,5,5′,5′-octamethyl-2,2′-bi(1,3,2-dioxaborolane) (303 mg, 1.2 mmol), Pd(dppf)Cl₂ (58 mg, 0.08 mmol) and potassium acetate (153 mg, 1.6 mmol), and the mixture was stirred at 90° C. for 3 hours. After cooling to room temperature, the mixture was concentrated under reduced pressure. The residue was dissolved in 1,4-dioxane (4.5 mL), added with A (100 mg, 0.3 mmol), Pd(dppf)Cl₂ (22 mg, 0.03 mmol) and potassium phosphate (140 mg, 0.6 mmol), and the reaction mixture was stirred at 90° C. overnight. The mixture was concentrated under reduced pressure, and the residue was purified by prep-HPLC to give the target product I-9 (40 mg, 31%).

MS m/z (ESI): 394.1 [M+H⁺]

¹H NMR (400 MHz, DMSO-d₆) δ 10.94 (s, 1H), 8.63 (ddd, J=4.9, 1.8, 0.8 Hz, 1H), 8.06-7.99 (m, 1H), 7.99-7.95 (m, 1H), 7.86 (d, J=8.1 Hz, 1H), 7.52 (ddd, J=7.4, 4.9, 1.0 Hz, 1H), 7.46-7.38 (m, 3H), 6.52-6.49 (m, 1H), 6.44 (dd, J=7.2, 1.9 Hz, 1H), 4.37 (dd, J=12.3, 5.0 Hz, 1H), 2.85-2.74 (m, 1H), 2.62-2.54 (m, 1H), 2.42-2.29 (m, 1H), 2.11-2.02 (m, 1H).

### Example 10

### 3-(2-Chloro-3-(3′-methyl-2-oxo-2H-[1,2′-bipyridin]-4-yl)phenyl)piperidine-2,6-dione (Compound I-10)

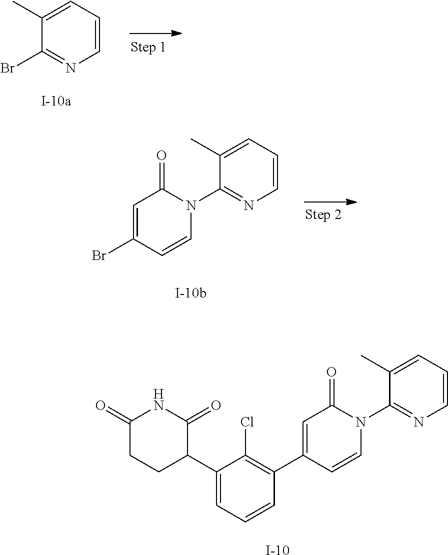

### Step 1

### 4-Bromo-3′-methyl-2H-[1,2′-bipyridin]-2-one (I-10b)

To a mixture of 2-iodo-3-methylpyridine I-10a (5 g, 23 mmol), 4-bromopyridin-2(1H)-one (4.0 g, 23 mmol), copper(I) iodide (434 mg, 2.3 mmol) and DMSO (100 mL) were added L-proline (263 mg, 2.3 mmol) and potassium carbonate (9.5 g, 69 mmol). The mixture was stirred at 100° C. for 5 hours. Water was added and the resulting mixture was extracted with ethyl acetate. The organic phase was concentrated under reduced pressure and the residue was purified by silica gel column chromatography (petroleum ether/ethyl acetate=1/1 v/v) to give the target product I-10b (50 mg, 1%).

MS m/z (ESI): 265.0 [M+H⁺]

### Step 2

### 3-(2-Chloro-3-(3′-methyl-2-oxo-2H-[1,2′-bipyridin]-4-yl)phenyl)piperidine-2,6-dione (I-10)

To a solution of I-10b (50 mg, 0.19 mmol) and B (66 mg, 0.19 mmol) in 1,4-dioxane (3 mL) were added bis(tri-tert-butylphosphine)palladium (10 mg, 0.019 mmol), DIPEA (73 mg, 0.57 mmol) and water (150 μL), and the reaction mixture was stirred at 100° C. for 3 hours. The mixture was concentrated under reduced pressure and the residue was purified by prep-HPLC to give the target product I-10 (12.0 mg, 16%).

MS m/z (ESI): 408.1 [M+H⁺]

¹H NMR (400 MHz, DMSO-d₆) δ 10.94 (s, 1H), 8.45 (dd, J=4.7, 1.2 Hz, 1H), 7.93-7.88 (m, 1H), 7.69 (d, J=7.0 Hz, 1H), 7.50 (dd, J=7.6, 4.8 Hz, 1H), 7.47-7.41 (m, 3H), 6.49 (s, 1H), 6.41 (dt, J=7.1, 1.8 Hz, 1H), 4.38 (dd, J=12.3, 5.0 Hz, 1H), 2.87-2.75 (m, 1H), 2.61-2.54 (m, 1H), 2.42-2.27 (m, 1H), 2.18 (s, 3H), 2.11-2.02 (m, 1H).

Compound I-23 was synthesized according to the procedures for Example 10, but a different reagent was used instead of I-10a.

The NMR data for compound 1-23 are as follows:

| Compound | ¹H NMR |
|---|---|
| 3-(2-chloro-3-(2-((5- | ¹H NMR (400 MHz, DMSO-d₆) δ 10.93 (s, 1H), 8.27 (d, J = 5.2 Hz, 1H), |
| cyclopropylpyridin-2- | 8.11 (d, J = 2.3 Hz, 1H), 7.56 (dd, J = 8.4, 2.5 Hz, 1H), 7.49-7.43 (m, |
| yl)oxy)pyridin-4- | 2H), 7.42-7.38 (m, 1H), 7.27-7.22 (m, 1H), 7.11 (s, 1H), 7.07 (d, J = |
| yl)phenyl)piperidine-2,6- | 8.4 Hz, 1H), 4.37 (dd, J = 12.2, 4.9 Hz, 1H), 2.85-2.74 (m, 1H), 2.63- |
| dione (I-23) | 2.54 (m, 1H), 2.42-2.30 (m, 1H), 2.09-2.02 (m, 1H), 2.01-1.93 (m, |
| | 1H), 1.03-0.95 (m, 2H), 0.75-0.69 (m, 2H). |

### Example 11

### 3-(2-Chloro-3-(5′-fluoro-2-oxo-2H-[1,2′-bipyridin]-4-yl)phenyl)piperidine-2,6-dione (Compound I-11)

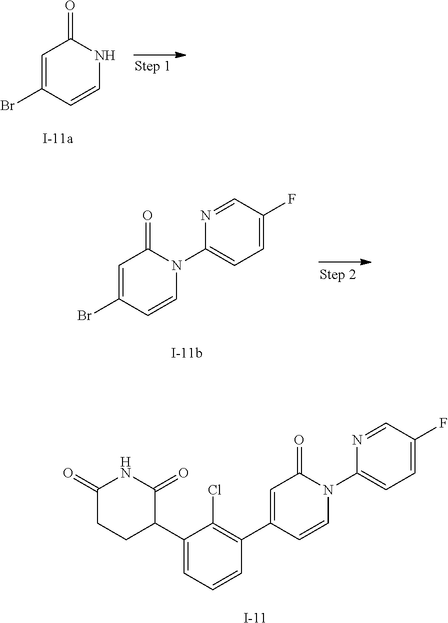

### Step 1

### 4-Bromo-5′-fluoro-2H-[1,2′-bipyridin]-2-one (I-11b)

To a solution of (5-fluoropyridin-2-yl)boronic acid (1 g, 7.1 mmol) and I-11a (1.2 g, 7.1 mmol) in 1,2-dichloroethane (10 mL) were added copper acetate (1.3 g, 7.1 mmol) and triethylamine (2.2 g, 21.3 mmol), and the reaction mixture was stirred at room temperature in air overnight. The mixture was concentrated under reduced pressure and the residue was purified by silica gel column chromatography (petroleum ether/ethyl acetate=86/14 v/v) to give the desired product I-11b (118 mg, 6%).

MS m/z (ESI): 269.0 [M+H⁺]

### Step 2

### 3-(2-Chloro-3-(5′-fluoro-2-oxo-2H-[1,2′-bipyridin]-4-yl)phenyl)piperidine-2,6-dione (I-11)

Compound I-11 was synthesized according to the procedure of Step 2 for Example 10, but I-11b was used instead of I-10b.

MS m/z (ESI): 412.0 [M+H⁺]

¹H NMR (400 MHz, DMSO-d₆) δ 10.93 (s, 1H), 8.64 (d, J=2.8 Hz, 1H), 8.03-7.89 (m, 3H), 7.52-7.37 (m, 3H), 6.52 (d, J=1.7 Hz, 1H), 6.45 (dd, J=7.2, 1.9 Hz, 1H), 4.37 (dd, J=12.3, 5.0 Hz, 1H), 2.87-2.75 (m, 1H), 2.64-2.56 (m, 1H), 2.43-2.28 (m, 1H), 2.11-2.01 (m, 1H).

### Example 12

### 3-(2-Chloro-3-(1-((1-cyclopropyl-1H-pyrazol-3-yl)methyl)-2-oxo-1,2-dihydropyridin-4-yl)phenyl)piperidine-2,6-dione (Compound I-12)

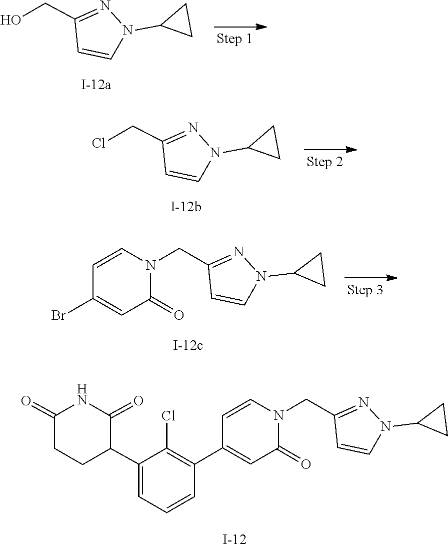

### Step 1

### 3-(Chloromethyl)-1-cyclopropyl-1H-pyrazole (I-12b)

A mixture of I-12a (250 mg, 1.8 mmol) and thionyl chloride (4 mL) was stirred at 70° C. for 3 hours. After cooling to room temperature, the reaction mixture was concentrated under reduced pressure. The residue was dissolved in ethyl acetate, added dropwise to a saturated aqueous solution of sodium bicarbonate and extracted with ethyl acetate. The organic phase was concentrated under reduced pressure to give the target product I-12b (crude, 271 mg). The product was used directly in the next step without further purification.

MS m/z (ESI): 157.1 [M+H⁺]

### Step 2

### 4-Bromo-1-((1-cyclopropyl-1H-pyrazol-3-yl)methyl)pyridin-2(1H)-one (I-12c)

To a solution of I-12b (271 mg, crude) and 4-bromopyridin-2(1H)-one (367 mg, 2.1 mmol) in DMF (8 mL) was added potassium carbonate (478 mg, 3.5 mmol), and the reaction mixture was stirred at 80° C. for 3 hours. Water was added and the mixture was extracted with ethyl acetate. The organic phase was concentrated under reduced pressure and the residue was purified by silica gel column chromatography (petroleum ether/ethyl acetate=100/0 to 30/70 v/v) to give the target product I-12c (280 mg, 55% for two steps).

MS m/z (ESI): 294.0 [M+H⁺]

### Step 3

### 3-(2-Chloro-3-(1-((1-cyclopropyl-1H-pyrazol-3-yl)methyl)-2-oxo-1,2-dihydropyridin-4-yl)phenyl)piperidine-2,6-dione (I-12)

To a solution of I-12c (51 mg, 0.17 mmol) and B (50 mg, 0.14 mmol) in 1,4-dioxane (10 mL) were added potassium phosphate (61 mg, 0.28 mmol) and Pd(dppf)Cl₂ (11 mg, 0.014 mmol), and the mixture was stirred at 90° C. overnight. The mixture was concentrated under reduced pressure and the residue was purified by prep-HPLC to give the target product I-12 (29 mg, 47%).

MS m/z (ESI): 437.2 [M+H⁺]

¹H NMR (400 MHz, DMSO-d₆) δ 10.92 (s, 1H), 7.76-7.67 (m, 2H), 7.45-7.37 (m, 2H), 7.33 (t, J=4.7 Hz, 1H), 6.37 (d, J=1.8 Hz, 1H), 6.27 (dd, J=7.0, 2.0 Hz, 1H), 6.17 (d, J=2.3 Hz, 1H), 5.05 (s, 2H), 4.34 (dd, J=12.3, 5.0 Hz, 1H), 3.72-3.64 (m, 1H), 2.86-2.74 (m, 1H), 2.59-2.53 (m, 1H), 2.38-2.25 (m, 1H), 2.08-1.95 (m, 1H), 1.04-0.90 (m, 4H).

### Example 13

### 3-(2-Chloro-3-(1-((1-cyclopropyl-1H-pyrazol-4-yl)methyl)-2-oxo-1,2-dihydropyridin-4-yl)phenyl)piperidine-2,6-dione (Compound I-13)

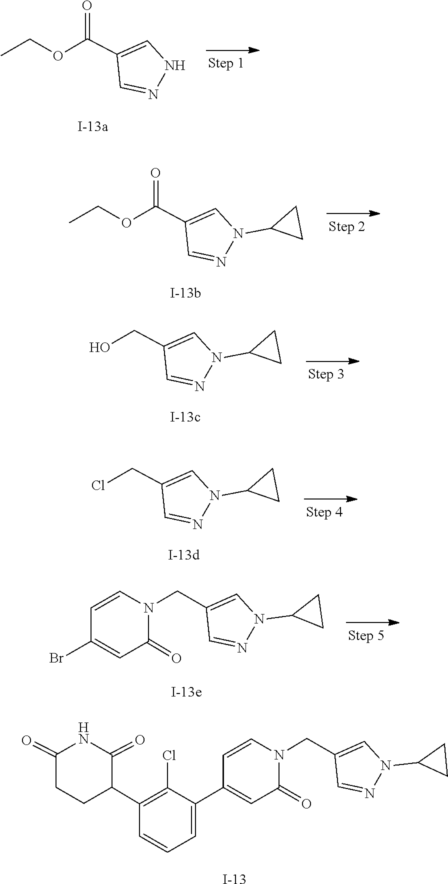

### Step 1

### Ethyl 1-cyclopropyl-1H-pyrazole-4-carboxylate (I-13b)

To a solution of I-13a (5.0 g, 36 mmol) and cyclopropylboronic acid (5.4 g, 62 mmol) in 1,2-dichloroethane (30 mL) were added copper acetate (7.8 g, 64 mmol), sodium bicarbonate (6.0 g, 71 mmol) and 2,2′-bipyridine (6.7 g, 43 mmol), and the reaction mixture was stirred at 70° C. in air overnight. An aqueous solution of sodium bicarbonate was added, and the resulting mixture was extracted with ethyl acetate. The organic phase was concentrated under reduced pressure and the residue was purified by silica gel column chromatography (petroleum ether/ethyl acetate=100/0 to 95/5 v/v) to give the target product I-13b (2.8 g, 44%).

MS m/z (ESI): 181.1 [M+H⁺]

### Step 2

### (1-Cyclopropyl-1H-pyrazol-4-yl)methanol (I-13c)

To a solution of I-13b (1.0 g, 5.5 mmol) in THF (10 mL) was added a solution of LiAlH₄ in THF (0.5 M, 33 mL, 17 mmol), and the reaction mixture was stirred at 0° C. for 2 hours. After quenching with 10% aqueous solution of sodium hydroxide, the resulting mixture was extracted with ethyl acetate. The organic phase was concentrated under reduced pressure to give the target product I-13c (crude, 400 mg). The product was used directly in the next step without further purification.

MS m/z (ESI): 139.1 [M+H⁺]

### Steps 3-5

### 3-(2-Chloro-3-(1-((1-cyclopropyl-1H-pyrazol-4-yl)methyl)-2-oxo-1,2-dihydropyridin-4-yl)phenyl)piperidine-2,6-dione (I-13)

Compound 1-13 was synthesized according to the procedures of Steps 1-3 for Example 12, but I-13c was used instead of I-12a.

MS m/z (ESI): 437.1 [M+H⁺]

¹H NMR (400 MHz, DMSO-d₆) δ 10.91 (s, 1H), 7.86-7.78 (m, 2H), 7.47 (s, 1H), 7.43-7.37 (m, 2H), 7.32 (t, J=4.6 Hz, 1H), 6.35 (d, J=1.8 Hz, 1H), 6.26 (dd, J=7.0, 1.9 Hz, 1H), 4.93 (s, 2H), 4.33 (dd, J=12.3, 5.0 Hz, 1H), 3.75-3.65 (m, 1H), 2.85-2.74 (m, 1H), 2.59-2.53 (m, 1H), 2.38-2.25 (m, 1H), 2.05-1.96 (m, 1H), 1.05-0.90 (m, 4H).

### Example 14

### 3-(2-Chloro-3-(1-((5-cyclopropylpyrimidin-2-yl)methyl)-2-oxo-1,2-dihydropyridin-4-yl)phenyl)piperidine-2,6-dione (Compound I-14)

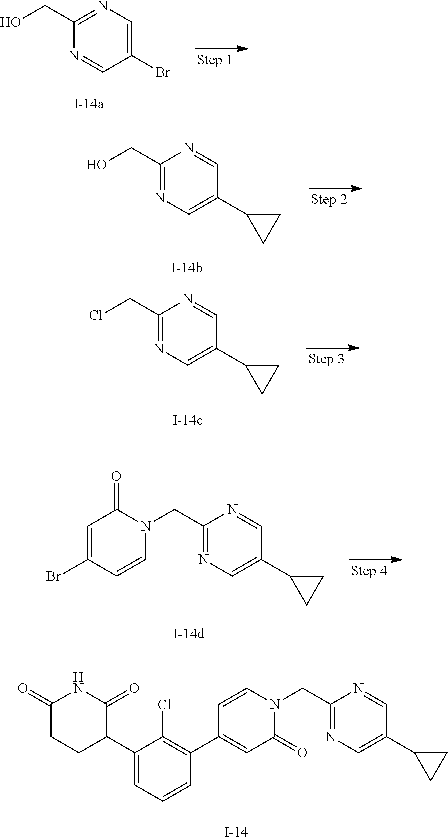

### Step 1

### (5-Cyclopropylpyrimidin-2-yl)methanol (I-14b)

To a mixture of (5-bromopyrimidin-2-yl)methanol I-14a (1.0 g, 5.3 mmol), cyclopropylboronic acid (910 mg, 10.6 mmol), potassium phosphate (3.37 g, 15.9 mmol), toluene (8 mL) and water (2 mL) were added triphenylphosphine (139 mg, 0.53 mmol) and Pd(OAc)₂ (60 mg, 0.27 mmol), and the mixture was stirred at 100° C. for 4 hours. The mixture was concentrated under reduced pressure and the residue was purified by silica gel column chromatography (dichloromethane/methanol=100/0 to 97/3 v/v) to give the target product I-14b (730 mg, 92%).

MS m/z (ESI): 151.1 [M+H⁺]

### Step 2

### 2-(Chloromethyl)-5-cyclopropylpyrimidine (I-14c)

A mixture of I-14b (700 mg, 4.7 mmol), thionyl chloride (2 mL) and dichloromethane (10 mL) was stirred at 70° C. for 1 hour. It was concentrated under reduced pressure. The residue was adjusted with saturated sodium bicarbonate solution to pH=7 and extracted with ethyl acetate. The organic phase was concentrated under reduced pressure and the residue was triturated with ether to yield the target product I-14c (415 mg, 53%).

MS m/z (ESI): 169.0 [M+H⁺]

### Step 3

### 4-Bromo-1-((5-cyclopropylpyrimidin-2-yl)methyl)pyridin-2(1H)-one (I-14d)

I-14d was synthesized according to the procedure of Step 1 for Example 3, but I-14c was used instead of I-2a.

MS m/z (ESI): 306.0 [M+H⁺]

### Step 4

### 3-(2-chloro-3-(1-((5-cyclopropylpyrimidin-2-yl)methyl)-2-oxo-1,2-dihydropyridin-4-yl)phenyl)piperidine-2,6-dione (I-14)

Compound I-14 was synthesized according to the procedure of Step 2 for Example 4, but I-14d was used instead of I-3b.

MS m/z (ESI): 449.1 [M+H⁺]

¹H NMR (400 MHz, DMSO-d₆) δ 10.93 (s, 1H), 8.54 (s, 2H), 7.83 (d, J=7.0 Hz, 1H), 7.47-7.41 (m, 2H), 7.40-7.34 (m, 1H), 6.37 (d, J=1.8 Hz, 1H), 6.33 (dd, J=7.0, 2.0 Hz, 1H), 5.29 (s, 2H), 4.36 (dd, J=12.2, 5.0 Hz, 1H), 2.86-2.74 (m, 1H), 2.59-2.54 (m, 1H), 2.41-2.27 (m, 1H), 2.10-2.01 (m, 1H), 1.97-1.88 (m, 1H), 1.06-0.98 (m, 2H), 0.86-0.78 (m, 2H).

### Example 15

### 3-(2-Chloro-3-(1-((6-cyclopropylpyridin-3-yl)methyl)-2-oxo-1,2-dihydropyridin-4-yl)phenyl)piperidine-2,6-dione (Compound I-15)

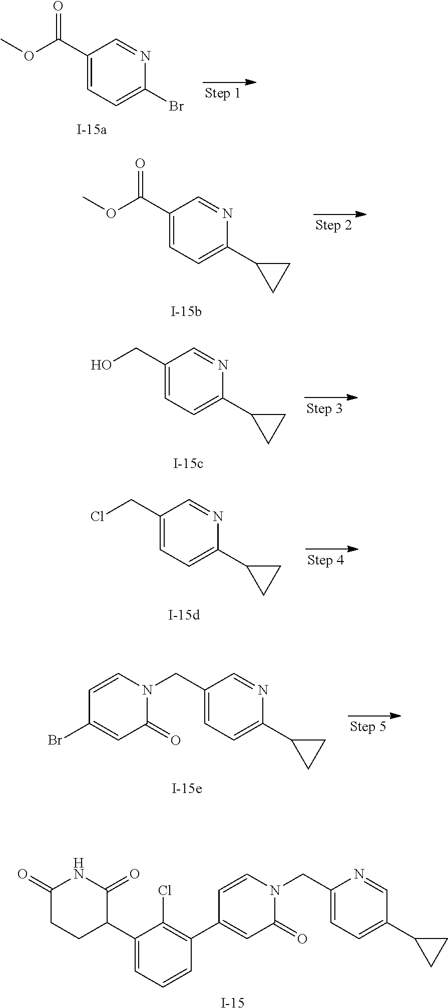

### Step 1

### Methyl 6-cyclopropylnicotinate (I-15b)

To a solution of cyclopropylboronic acid (1.2 g, 14.4 mmol) and I-15a (2.1 g, 9.6 mmol) in 1,4-dioxane (20 mL) were added Pd(dppf)Cl₂ (705 mg, 0.96 mmol), potassium phosphate (4.1 g, 19.3 mmol) and water (1 mL), and the mixture was stirred at 100° C. for 6 hours. The mixture was concentrated under reduced pressure and the residue was purified by silica gel column chromatography (petroleum ether/ethyl acetate=89/11 v/v) to give the target product I-15b (700 mg, 41%).

MS m/z (ESI): 178.1 [M+H⁺]

### Step 2

### (6-Cyclopropylpyridin-3-yl)methanol (I-15c)

A solution of LiAlH₄ in THF (2.5 M, 3.1 mL, 7.9 mmol) was added dropwise to a solution of I-15b (700 mg, 3.9 mmol) in THF (7 mL) at 0° C., and the reaction mixture was stirred at 0° C. for 1 hour. The reaction was quenched with saturated ammonium chloride solution and the mixture was extracted with ethyl acetate. The organic phase was dried over anhydrous sodium sulfate and concentrated under reduced pressure to give the target product I-15c (548 mg, 93%).

MS m/z (ESI): 150.1 [M+H⁺]

### Step 3

### 5-(Chloromethyl)-2-cyclopropylpyridine (I-15d)

I-15c (548 mg, 3.7 mmol) was dissolved in thionyl chloride (4 mL), and the resulting mixture was stirred at 70° C. for 2 hours. The mixture was concentrated under reduced pressure, added with saturated sodium carbonate solution and ethyl acetate. The organic layer was separated, dried over anhydrous sodium sulfate and concentrated under reduced pressure to give the target product I-15d (453 mg, 74%).

MS m/z (ESI): 168.0 [M+H⁺]

### Step 4

### 4-Bromo-1-((6-cyclopropylpyridin-3-yl)methyl)pyridin-2(1H)-one (I-15e)

To a solution of I-15d (453 mg, 2.7 mmol) and 4-bromopyridin-2(1H)-one (563 mg, 3.2 mmol) in DMA (8 mL) was added potassium carbonate (1.1 g, 8.1 mmol), and the mixture was stirred at room temperature overnight. After quenching with water, the mixture was extracted with ethyl acetate. The organic phase was dried over anhydrous sodium sulfate and concentrated under reduced pressure. The residue was purified by silica gel column chromatography (petroleum ether/ethyl acetate=63/37 v/v) to give the target product I-15e (553 mg, 67%).

MS m/z (ESI): 305.0 [M+H⁺]

### Step 5

### 3-(2-Chloro-3-(1-((6-cyclopropylpyridin-3-yl)methyl)-2-oxo-1,2-dihydropyridin-4-yl)phenyl)piperidine-2,6-dione (I-15)

I-15 was synthesized according to the procedure of Step 2 for Example 10, but I-15e was used instead of I-10b.

MS m/z (ESI): 448.2 [M+H⁺]

¹H NMR (400 MHz, DMSO-d₆) δ 10.91 (s, 1H), 8.46 (d, J=1.9 Hz, 1H), 7.92 (d, J=7.0 Hz, 1H), 7.66 (dd, J=8.1, 2.3 Hz, 1H), 7.46-7.38 (m, 2H), 7.38-7.31 (m, 1H), 7.28 (d, J=8.1 Hz, 1H), 6.39 (d, J=1.8 Hz, 1H), 6.31 (dd, J=7.0, 2.0 Hz, 1H), 5.09 (s, 2H), 4.34 (dd, J=12.3, 5.0 Hz, 1H), 2.85-2.73 (m, 1H), 2.58-2.53 (m, 1H), 2.38-2.26 (m, 1H), 2.12-1.99 (m, 2H), 0.97-0.84 (m, 4H).

### Example 16

### 3-(2-Chloro-3-(1-(1-(5-cyclopropylpyridin-2-yl)ethyl)-2-oxo-1,2-dihydropyridin-4-yl)phenyl)piperidine-2,6-dione (Compound I-16)

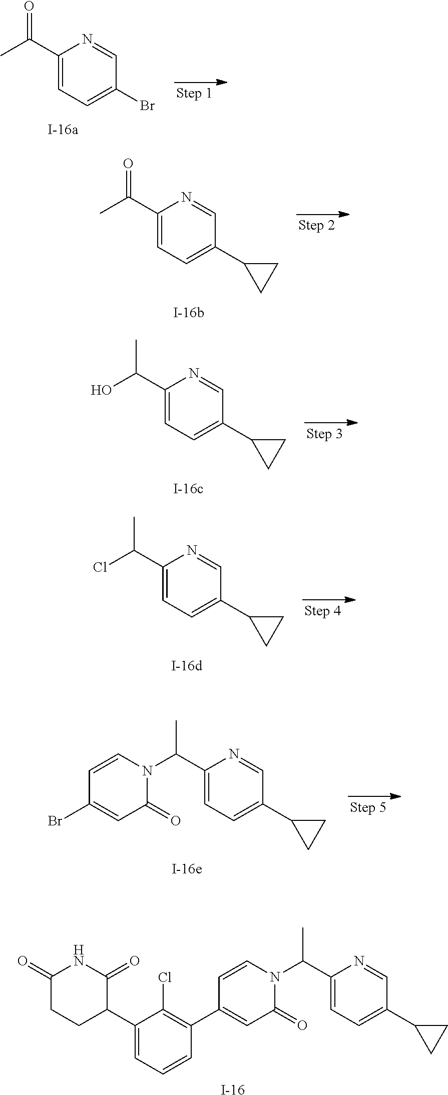

### Step 1

### 1-(5-Cyclopropylpyridin-2-yl)ethan-1-one (I-16b)

I-16b was synthesized according to the procedure of Step 1 for Example 15, but I-16a was used instead of I-15a.

MS m/z (ESI): 162.1 [M+H⁺]

### Step 2

### 1-(5-Cyclopropylpyridin-2-yl)ethan-1-ol (I-16c)

To a solution of I-16b (2.1 g, 13.0 mmol) in methanol (20 mL) was added sodium borohydride (990 mg, 26.0 mmol), and the reaction mixture was stirred at room temperature for 2 hours. The mixture was concentrated under reduced pressure, added with water and extracted with dichloromethane. The organic phase was concentrated under reduced pressure and the residue was purified by silica gel column chromatography (dichloromethane/methanol=3/2 v/v) to give the target product I-16c (2 g, 94%).

MS m/z (ESI): 164.1 [M+H⁺]

### Steps 3-4

### 4-Bromo-1-(1-(5-cyclopropylpyridin-2-yl)ethyl)pyridin-2(1H)-one (I-16e)

I-16e was synthesized according to the procedures of Steps 1-2 for Example 12, but I-16c was used instead of I-12a.

MS m/z (ESI): 319.0 [M+H⁺]

### Step 5

### 3-(2-Chloro-3-(1-(1-(5-cyclopropylpyridin-2-yl)ethyl)-2-oxo-1,2-dihydropyridin-4-yl)phenyl)piperidine-2,6-dione (I-16)

I-16 was synthesized according to the procedure of Step 2 for Example 10, but I-16e was used instead of I-10b.

MS m/z (ESI): 462.1 [M+H⁺]

¹H NMR (400 MHz, DMSO-d₆) δ 10.92 (s, 1H), 8.40 (d, J=2.1 Hz, 1H), 7.76 (d, J=7.2 Hz, 1H), 7.47-7.37 (m, 3H), 7.34 (t, J=4.7 Hz, 1H), 7.27 (d, J=8.1 Hz, 1H), 6.37 (s, 1H), 6.30 (d, J=7.2 Hz, 1H), 6.17 (q, J=7.1 Hz, 1H), 4.34 (dd, J=12.2, 5.0 Hz, 1H), 2.86-2.73 (m, 1H), 2.59-2.53 (m, 1H), 2.38-2.25 (m, 1H), 2.08-1.99 (m, 1H), 1.98-1.90 (m, 1H), 1.71 (d, J=7.1 Hz, 3H), 1.03-0.96 (m, 2H), 0.77-0.69 (m, 2H).

### Example 17

### 3-(2-Chloro-3-(1-((5-fluoropyridin-2-yl)methyl)-2-oxo-1,2-dihydropyridin-4-yl)phenyl)piperidine-2,6-dione (Compound I-17)

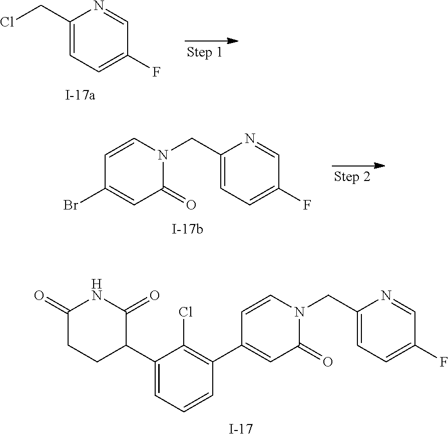

### Step 1

### 4-Bromo-1-((5-fluoropyridin-2-yl)methyl)pyridin-2(1H)-one (I-17b)

I-17b was synthesized according to the procedure of Step 1 for Example 7, but I-17a was used instead of I-7a.

MS m/z (ESI): 283.0 [M+H⁺]

### Step 2

### 3-(2-Chloro-3-(1-((5-fluoropyridin-2-yl)methyl)-2-oxo-1,2-dihydropyridin-4-yl)phenyl)piperidine-2,6-dione (I-17)

I-17 was synthesized according to the procedure of Step 3 for Example 12, but I-17b was used instead of I-12c.

MS m/z (ESI): 426.1 [M+H⁺]

¹H NMR (400 MHz, DMSO-d₆) δ 10.92 (s, 1H), 8.54 (d, J=2.9 Hz, 1H), 7.86 (d, J=7.0 Hz, 1H), 7.74 (td, J=8.7, 3.0 Hz, 1H), 7.47-7.40 (m, 3H), 7.39-7.32 (m, 1H), 6.38 (d, J=1.8 Hz, 1H), 6.33 (dd, J=7.0, 2.0 Hz, 1H), 5.22 (s, 2H), 4.35 (dd, J=12.2, 5.0 Hz, 1H), 2.86-2.75 (m, 1H), 2.62-2.54 (m, 1H), 2.41-2.27 (m, 1H), 2.08-2.00 (m, 1H).

Compound I-19 was synthesized according to the procedures for Example 17, but a different reagent was used instead of I-17a.

| Compound | | Compound replacing | MS m/z |
|---|---|---|---|
| Number | Compound Structure | I-17a | (ESI) |
| I-19 | 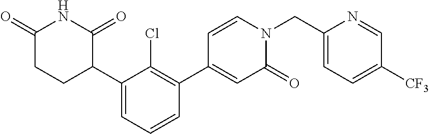 [SMILES: O=C1CCC(c2cccc(-c3ccn(Cc4ccc(C(F)(F)F)cn4)c(=O)c3)c2Cl)C(=O)N1] | 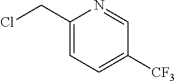 [SMILES: FC(F)(F)c1ccc(CCl)nc1] | 476.2 [M + H⁺] |

The NMR data for compound I-19 are as follows:

| Compound | ¹H NMR |
|---|---|
| 3-(2-chloro-3-(2-oxo-1-((5- | ¹H NMR (400 MHz, DMSO-d₆) δ 10.93 (s, 1H), 8.94 (s, 1H), 8.23 (dd, J = 8.2, |
| (trifluoromethyl)pyridin-2- | 2.0 Hz, 1H), 7.90 (d, J = 7.0 Hz, 1H), 7.56 (d, J = 8.2 Hz, 1H), 7.46-7.33 (m, |
| yl)methyl)-1,2-dihydropyridin-4- | 3H), 6.40 (d, J = 1.6 Hz, 1H), 6.37 (dd, J = 7.0, 1.9 Hz, 1H), 5.34 (s, 2H), 4.35 |
| yl)phenyl)piperidine-2,6-dione | (dd, J = 12.2, 5.0 Hz, 1H), 2.85-2.75 (m, 1H), 2.59-2.53 (m, 1H), 2.40-2.28 |
| (I-19) | (m, 1H), 2.09-2.00 (m, 1H). |

### Example 18

### 3-(2-Chloro-3-(1-((5-cyclopropoxypyridin-2-yl)methyl)-2-oxo-1,2-dihydropyridin-4-yl)phenyl)piperidine-2,6-dione (Compound 1-18)

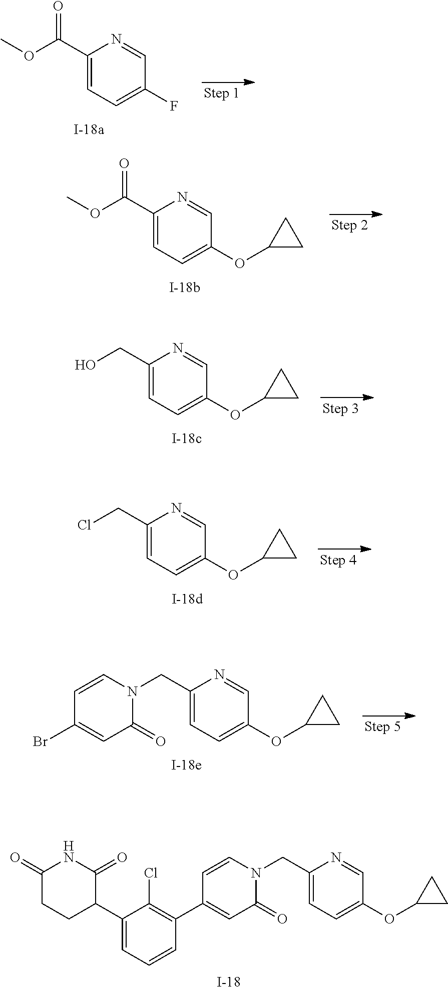

### Step 1

### Methyl 5-cyclopropoxypicolinate (I-18b)

To a solution of methyl 5-fluoropyridine-2-carboxylate I-18a (4 g, 25.8 mmol) and cyclopropanol (2.3 g, 39 mmol) in THF (50 mL) was added sodium hydride (60%, 2.1 g, 52 mmol), and the mixture was stirred at room temperature for 3 hours. The reaction was quenched with water and the mixture was extracted with ethyl acetate. The organic phase was concentrated under reduced pressure and the residue was purified by silica gel column chromatography (petroleum ether/ethyl acetate=3/1 v/v) to give the target product I-18b (2.5 g, 50%).

MS m/z (ESI): 194.0 [M+H⁺]

### Step 2

### (5-Cyclopropyloxypyridin-2-yl)methanol (I-18c)

I-18c was synthesized according to the procedure of Step 2 for Example 15, but I-18b was used instead of I-15b.

MS m/z (ESI): 166.1 [M+H⁺]

### Step 3-4

### 4-Bromo-1-((5-cyclopropoxypyridin-2-yl)methyl)pyridin-2(1H)-one (I-18e)

I-18e was synthesized according to the procedures of Steps 1-2 for Example 12, but I-18c was used instead of I-12a.

MS m/z (ESI): 321.0 [M+H⁺]

### Step 5

### 3-(2-Chloro-3-(1-((5-cyclopropoxypyridin-2-yl)methyl)-2-oxo-1,2-dihydropyridin-4-yl)phenyl)piperidine-2,6-dione (I-18)

I-18 was synthesized according to the procedure of Step 2 for Example 10, but I-18e was used instead of I-10b.

MS m/z (ESI): 464.1 [M+H⁺]

¹H NMR (400 MHz, DMSO-d₆) δ 10.92 (s, 1H), 8.32 (d, J=2.6 Hz, 1H), 7.83 (d, J=7.0 Hz, 1H), 7.52 (dd, J=8.5, 2.3 Hz, 1H), 7.43-7.39 (m, 2H), 7.37-7.30 (m, 2H), 6.37 (s, 1H), 6.31 (d, J=7.0 Hz, 1H), 5.16 (s, 2H), 4.35 (dd, J=12.2, 4.9 Hz, 1H), 3.97-3.89 (m, 1H), 2.86-2.72 (m, 1H), 2.62-2.54 (m, 1H), 2.38-2.26 (m, 1H), 2.17-1.99 (m, 1H), 0.84-0.77 (m, 2H), 0.72-0.65 (m, 2H).

### Example 19

### 3-(2-Chloro-3-(1-((6-cyclopropylpyridazin-3-yl)methyl)-2-oxo-1,2-dihydropyridin-4-yl)phenyl)piperidine-2,6-dione (Compound I-20)

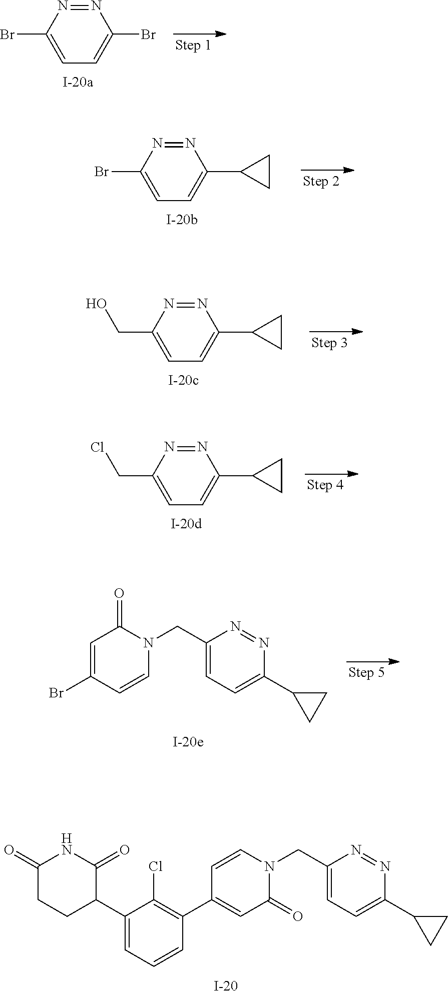

### Step 1

### 3-Bromo-6-cyclopropylpyridazine (I-20b)

I-20b was synthesized according to the procedure of Step 1 for Example 15, but I-20a was used instead of I-15a.

MS m/z (ESI): 199.0 [M+H⁺]

### Step 2

### (6-cyclopropylpyridazin-3-yl)methanol (I-20c)

To a solution of I-20b (600 mg, 3.0 mmol) and tributyltin methanol (1.9 g, 6.0 mmol) in 1,4-dioxane (10 mL) was added Pd(PPh₃)₄ (347 mg, 0.3 mmol), and the reaction mixture was stirred at 100° C. for 12 hours. The mixture was concentrated under reduced pressure and the residue was purified by silica gel column chromatography (dichloromethane/methanol=25/1 v/v) to give the target product I-20c (110 mg, 24%).

MS m/z (ESI): 151.1 [M+H⁺]

### Step 3

### 3-(Chloromethyl)-6-cyclopropylpyridazine (I-20d)

A mixture of I-20c (100 mg, 0.67 mmol) and thionyl chloride (2 mL) was stirred at room temperature for 2 hours. After quenching with ice water, the mixture was neutralized with saturated sodium bicarbonate solution and extracted with dichloromethane. The organic phase was concentrated under reduced pressure to give the target product I-20d (100 mg, 89%). The product was used directly in the next step without further purification.

MS m/z (ESI): 169.0 [M+H⁺]

### Step 4

### 4-Bromo-1-((6-cyclopropylpyridazin-3-yl)methyl)pyridin-2(1H)-one (I-20e)

I-20e was synthesized according to the procedure of Step 2 for Example 12, but I-20d was used instead of I-12b.

MS m/z (ESI): 306.0 [M+H⁺]

### Step 5

### 3-(2-Chloro-3-(1-((6-cyclopropylpyridazin-3-yl)methyl)-2-oxo-1,2-dihydropyridin-4-yl)phenyl)piperidine-2,6-dione (I-20)

To a solution of I-20e (60 mg, 0.2 mmol) and B (69 mg, 0.2 mmol) in DMF (4 mL) were added potassium phosphate (127 mg, 0.6 mmol) and Pd(dppf)Cl₂ (15 mg, 0.02 mmol). The reaction mixture was stirred at 100° C. for 3 hours and concentrated under reduced pressure. The residue was purified by prep-HPLC to give the target product I-20 (28 mg, 32%).

MS m/z (ESI): 449.1 [M+H⁺]

¹H NMR (400 MHz, DMSO-d₆) δ 10.92 (s, 1H), 7.90 (d, J=7.0 Hz, 1H), 7.51 (s, 2H), 7.44-7.39 (m, 2H), 7.38-7.33 (m, 1H), 6.39 (d, J=1.7 Hz, 1H), 6.35 (dd, J=7.0, 1.9 Hz, 1H), 5.35 (s, 2H), 4.35 (dd, J=12.3, 5.0 Hz, 1H), 2.85-2.75 (m, 1H), 2.61-2.53 (m, 1H), 2.39-2.19 (m, 2H), 2.08-1.99 (m, 1H), 1.12-0.98 (m, 4H).

### Example 20

### 3-(2-Chloro-3-(1-((5-cyclopropylpyridin-2-yl)methyl)-2-oxo-1,2-dihydropyridin-4-yl)phenyl)-3-fluoropiperidine-2,6-dione (Compound I-21)

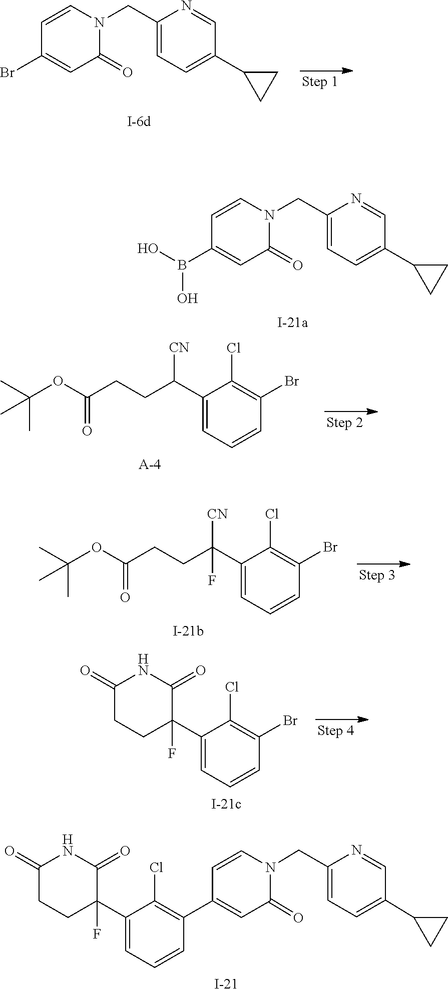

### Step 1

### (1-((5-Cyclopropylpyridin-2-yl)methyl)-2-oxo-1,2-dihydropyridin-4-yl)boronic acid (I-21a)

To a solution of I-6d (200 mg, 0.73 mmol) in 1,4-dioxane (10 mL) was added 4,4,4′,4′,5,5,5′,5′-octamethyl-2,2′-bi(1,3,2-dioxaborolane) (220 mg, 0.87 mmol), Pd(dppf)Cl₂ (54 mg, 0.073 mmol) and potassium acetate (142 mg, 1.5 mmol), and the mixture was stirred at 90° C. for 3 hours. After cooling to room temperature, the mixture was concentrated under reduced pressure and the residue was purified by silica gel column chromatography (petroleum ether/ethyl acetate=45/55 v/v), followed by further purification using prep-HPLC to give the target product I-21a (100 mg, 56%).

MS m/z (ESI): 271.1 [M+H⁺]

### Step 2

### tert-Butyl 4-(3-bromo-2-chlorophenyl)-4-cyano-4-fluorobutanoate (I-21b)

To a solution of A-4 (500 mg, 1.4 mmol) in THF (5 mL) was added a solution of NaHMDS in THF (2 M, 1.05 mL, 2.1 mmol), and the mixture was stirred at −78° C. for 1 hour. NFSI (659 mg, 2.1 mmol) was added, and the mixture was slowly warmed to 0° C. and stirred for 1 hour. After quenching with saturated ammonium chloride solution, the mixture was extracted with ethyl acetate. The organic phase was concentrated under reduced pressure and the residue was purified by silica gel column chromatography (hexane/ethyl acetate=100/0 to 40/60 v/v) to give the target product I-21b (300 mg, 57%).

MS m/z (ESI): 376.1 [M+H⁺]

### Step 3

### 3-(3-Bromo-2-chlorophenyl)-3-fluoropiperidine-2,6-dione (I-21c)

To a solution of I-21b (300 mg, 0.80 mmol) in acetic acid (2 mL) was added concentrated sulfuric acid (0.2 mL), and the resulting mixture was stirred at 90° C. for 3 hours. The mixture was concentrated under reduced pressure. The residue was added with ethyl acetate and filtered. The filter cake was washed with ethyl acetate to give the target product I-21c (crude, 60 mg).

MS m/z (ESI): 320.0 [M+H⁺]

### Step 4

### 3-(2-Chloro-3-(1-((5-cyclopropylpyridin-2-yl)methyl)-2-oxo-1,2-dihydropyridin-4-yl)phenyl)-3-fluoropiperidine-2,6-dione (I-21)

Compound I-21 was synthesized according to the procedure of Step 2 for Example 10, but I-21c was used instead of I-10b and I-21a was used instead of B.

MS m/z (ESI): 466.2 [M+H⁺]

¹H NMR (400 MHz, DMSO-d₆) δ 11.53 (s, 1H), 8.35 (d, J=2.0 Hz, 1H), 7.84 (d, J=7.0 Hz, 1H), 7.72 (d, J=8.2 Hz, 1H), 7.59 (t, J=7.7 Hz, 1H), 7.54-7.46 (m, 1H), 7.42 (dd, J=8.1, 2.3 Hz, 1H), 7.20 (d, J=8.1 Hz, 1H), 6.38 (d, J=1.7 Hz, 1H), 6.30 (dd, J=7.0, 1.9 Hz, 1H), 5.16 (s, 2H), 2.96-2.83 (m, 1H), 2.67-2.58 (m, 1H), 2.38-2.24 (m, 1H), 2.04-1.88 (m, 2H), 1.02-0.97 (m, 2H), 0.74-0.68 (m, 2H).

### Example 21

### 3-(2-Chloro-3-(2-(pyridin-2-yloxy)pyridin-4-yl)phenyl)piperidine-2,6-dione (Compound I-22)

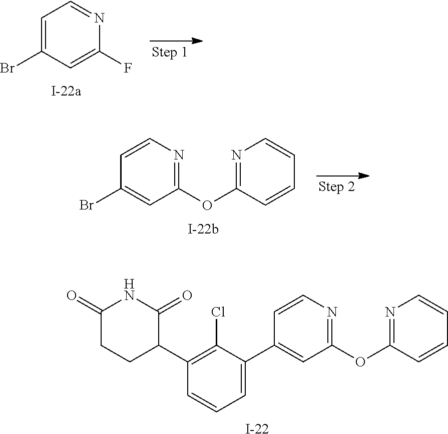

### Step 1

### 4-Bromo-2-(pyridin-2-yloxy)pyridine (I-22b)

To a solution of pyridine-2-boronic acid (965 mg, 7.9 mmol) and 4-bromopyridin-2-ol I-22a (1.4 g, 7.9 mmol) in 1,2-dichloroethane (10 mL) were added copper acetate (1.4 g, 7.9 mmol) and triethylamine (2.4 g, 23.6 mmol), and the reaction mixture was stirred at room temperature in air overnight. The mixture was concentrated under reduced pressure and the residue was purified by silica gel column chromatography (petroleum ether/ethyl acetate=81/19 v/v) to give the target product I-22b (20 mg, 1%).

MS m/z (ESI): 250.9 [M+H⁺]

### Step 2

### 3-(2-Chloro-3-(2-(pyridin-2-yloxy)pyridin-4-yl)phenyl)piperidine-2,6-dione (I-22)

Compound I-22 was synthesized according to the procedure of Step 2 for Example 10, but I-22b was used instead of I-10b.

MS m/z (ESI): 394.0 [M+H⁺]

¹H NMR (400 MHz, DMSO-d₆) δ 10.93 (s, 1H), 8.31 (d, J=5.1 Hz, 1H), 8.29-8.25 (m, 1H), 7.95-7.89 (m, 1H), 7.48-7.38 (m, 3H), 7.29 (d, J=5.2 Hz, 1H), 7.27-7.23 (m, 1H), 7.21-7.15 (m, 2H), 4.37 (dd, J=12.2, 4.9 Hz, 1H), 2.87-2.75 (m, 1H), 2.59-2.54 (m, 1H), 2.41-2.28 (m, 1H), 2.10-1.99 (m, 1H).

### Example 22

### 3-(2-Chloro-3-(2-((6-cyclopropylpyridin-3-yl)oxy)pyridin-4-yl)phenyl)piperidine-2,6-dione (Compound I-24)

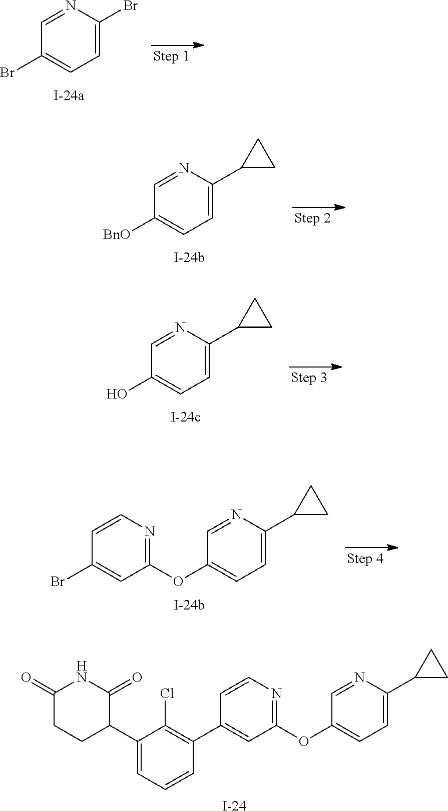

### Step 1

### 5-(Benzyloxy)-2-cyclopropylpyridine (I-24b)

To a solution of cyclopropylboronic acid (665 mg, 7.7 mmol) and 5-(benzyloxy)-2-bromopyridine I-24a (1.4 g, 5.2 mmol) in toluene (20 mL) were added potassium phosphate (3.8 g, 18 mmol), tricyclohexylphosphine (144 mg, 0.52 mmol), palladium acetate (58 mg, 0.26 mmol) and water (4 mL). The mixture was stirred at 100° C. for 8 hours and concentrated under reduced pressure. The residue was purified by silica gel column chromatography (petroleum ether/ethyl acetate=94/6 v/v) to give the target product I-24b (296 mg, 25%).

MS m/z (ESI): 226.1 [M+H⁺]

### Step 2

### 6-Cyclopropylpyridin-3-ol (I-24c)

To a solution of I-24b (296 mg, 1.3 mmol) in methanol (8 mL) was added palladium hydroxide on carbon (100 mg), and the mixture was stirred at room temperature under a hydrogen atmosphere for 1.5 hours. The mixture was filtered, and the filtrate was concentrated under reduced pressure to give the target product I-24c (crude, 161 mg). The product was used directly in the next step without further purification.

MS m/z (ESI): 136.2 [M+H⁺]

### Step 3

### 4-Bromo-2-((6-cyclopropylpyridin-3-yl)oxy)pyridine (I-24d)

To a solution of I-24c (161 mg, 1.2 mmol) in THF (5 mL) was added sodium hydride (60%, 48 mg, 2.0 mmol), and the mixture was stirred at room temperature for 15 minutes. The mixture was added with 4-bromo-2-fluoropyridine (211 mg, 1.2 mmol) and stirred at 65° C. for 24 hours. After quenching with methanol, the mixture was concentrated under reduced pressure and the residue was purified by silica gel column chromatography (petroleum ether/ethyl acetate=95/5 v/v) to give the target product I-24d (62 mg, 18%).

MS m/z (ESI): 291.0 [M+H⁺]

### Step 4

### 3-(2-Chloro-3-(2-((6-cyclopropylpyridin-3-yl)oxy)pyridin-4-yl)phenyl)piperidine-2,6-dione (I-24)

Compound I-24 was synthesized according to the procedure of Step 2 for Example 10, but I-24d was used instead of I-10b.

MS m/z (ESI): 434.1 [M+H⁺]

¹H NMR (400 MHz, DMSO-d₆) δ 10.93 (s, 1H), 8.33 (d, J=2.7 Hz, 1H), 8.23-8.17 (m, 1H), 7.57 (dd, J=8.5, 2.7 Hz, 1H), 7.50-7.44 (m, 2H), 7.41-7.34 (m, 2H), 7.21 (dd, J=5.2, 1.4 Hz, 1H), 7.13 (s, 1H), 4.37 (dd, J=12.3, 5.0 Hz, 1H), 2.86-2.75 (m, 1H), 2.59-2.53 (m, 1H), 2.41-2.27 (m, 1H), 2.19-2.10 (m, 1H), 2.09-1.99 (m, 1H), 1.01-0.88 (m, 4H).

### Example 23

### 6-((4-(2-Chloro-3-(2,6-dioxopiperidin-3-yl)phenyl)-2-oxopyridin-1(2H)-yl)methyl)nicotinonitrile (Compound I-25)

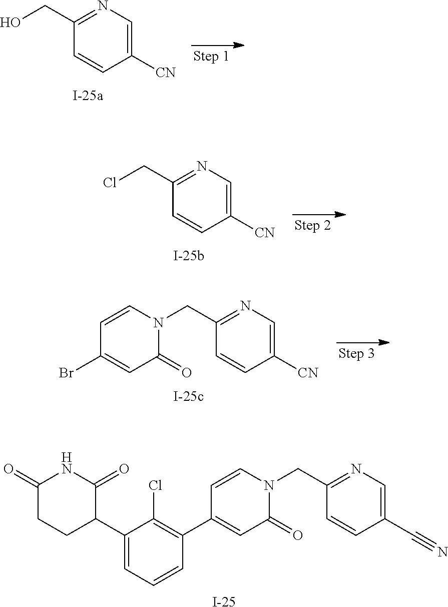

### Step 1

### 6-(Chloromethyl)nicotinonitrile (I-25b)

To a solution of 6-(hydroxymethyl)nicotinonitrile I-25a (110 mg, 0.82 mmol) in dichloromethane (5 mL) at 0° C. was added thionyl chloride (119 L, 1.6 mmol) slowly. The reaction mixture was stirred at room temperature for 2 hours, quenched with saturated sodium bicarbonate solution and extracted with dichloromethane. The organic phase was dried over anhydrous sodium sulfate, filtered and concentrated under reduced pressure to give the target product I-25b (crude, 140 mg). The product was used directly in the next step without further purification.

MS m/z (ESI): 153.0 [M+H⁺]

### Step 2

### 6-((4-Bromo-2-oxopyridin-1(2H)-yl)methyl)nicotinonitrile (I-25c)

I-25c was synthesized according to the procedure of Step 2 for Example 12, but I-25b was used instead of I-12b.

MS m/z (ESI): 289.9 [M+H⁺]

### Step 3

### 6-((4-(2-Chloro-3-(2,6-dioxopiperidin-3-yl)phenyl)-2-oxopyridin-1(2H)-yl)methyl)nicotinonitrile (I-25)

To a solution of I-25c (44 mg, 0.15 mmol) in DMF (5 mL) were added B (53 mg, 0.15 mmol), potassium phosphate (64.6 mg, 0.3 mmol) and Pd(dppf)Cl₂ (11.1 mg, 0.015 mmol), and the mixture was stirred at 100° C. for 3 hours. The mixture was added with water and extracted with ethyl acetate. The organic phase was concentrated under reduced pressure and the residue was purified by prep-HPLC to give the target product I-25 (15.8 mg, 24%).

MS m/z (ESI): 433.1 [M+H⁺]

¹H NMR (400 MHz, DMSO-d₆) δ 10.93 (s, 1H), 8.99 (d, J=1.3 Hz, 1H), 8.32 (dd, J=8.2, 2.1 Hz, 1H), 7.88 (d, J=7.0 Hz, 1H), 7.54 (d, J=8.2 Hz, 1H), 7.46-7.33 (m, 3H), 6.40 (d, J=1.4 Hz, 1H), 6.36 (dd, J=7.0, 1.8 Hz, 1H), 5.32 (s, 2H), 4.35 (dd, J=12.2, 5.0 Hz, 1H), 2.85-2.74 (m, 1H), 2.61-2.55 (m, 1H), 2.41-2.27 (m, 1H), 2.09-1.95 (m, 1H).

### Example 24

### 3-(2-Chloro-3-(2-((5-cyclopropylpyridin-2-yl)methoxy)pyridin-4-yl)phenyl)piperidine-2,6-dione (Compound I-27)

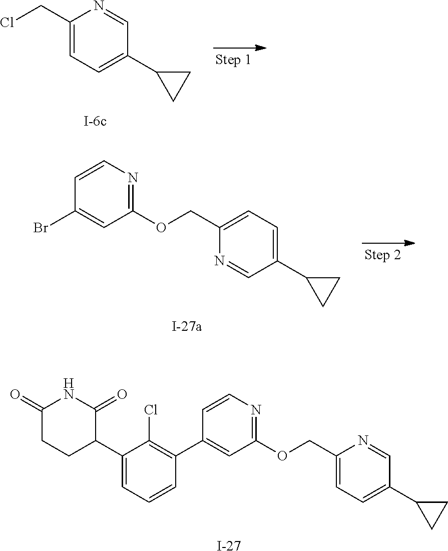

### Step 1

### 4-Bromo-2-((5-cyclopropylpyridin-2-yl)methoxy)pyridine (I-27a)

A mixture of I-6c (6 g, 34 mmol), 4-bromopyridin-2(1H)-one (5.9 g, 34 mmol), cesium carbonate (33.0 g, 103 mmol) and acetonitrile (60 mL) was stirred at 80° C. for 2 hours. The mixture was added with water and extracted with ethyl acetate. The organic phase was concentrated under reduced pressure and the residue was purified by silica gel column chromatography (petroleum ether/ethyl acetate=70/30 v/v) to give the target product I-27a (203 mg, 2%).

MS m/z (ESI): 305.1 [M+H⁺]

### Step 2

### 3-(2-Chloro-3-(2-((5-cyclopropylpyridin-2-yl)methoxy)pyridin-4-yl)phenyl)piperidine-2,6-dione (I-27)

Compound I-27 was synthesized according to the procedure of Step 3 for Example 12, but I-27a was used instead of I-12c.

MS m/z (ESI): 448.1 [M+H⁺]

¹H NMR (400 MHz, DMSO-d₆) δ 10.92 (s, 1H), 8.39 (d, J=1.9 Hz, 1H), 8.23 (d, J=5.2 Hz, 1H), 7.48-7.41 (m, 3H), 7.40-7.33 (m, 2H), 7.06 (dd, J=5.3, 1.4 Hz, 1H), 6.93 (s, 1H), 5.41 (s, 2H), 4.36 (dd, J=12.3, 5.1 Hz, 1H), 2.87-2.74 (m, 1H), 2.59-2.53 (m, 1H), 2.38-2.29 (m, 1H), 2.10-1.92 (m, 2H), 1.04-0.96 (m, 2H), 0.78-0.69 (m, 2H).

### Example 25

### 3-(2-Chloro-3-(2-oxo-2H-[1,3′-bipyridin]-4-yl)phenyl)piperidine-2,6-dione (Compound I-28)

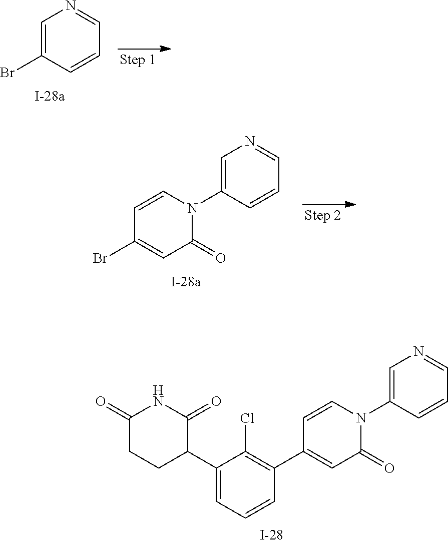

### Step 1

### 4-Bromo-2H-[1,3′-bipyridin]-2-one (I-28b)

I-28b was synthesized according to the procedure of Step 1 for Example 9, but I-28a was used instead of I-9a.

MS m/z (ESI): 251.0 [M+H⁺]

### Step 2

### 3-(2-Chloro-3-(2-oxo-2H-[1,3′-bipyridin]-4-yl)phenyl)piperidine-2,6-dione (I-28)

Compound I-28 was synthesized according to the procedure of Step 3 for Example 12, but I-28b was used instead of I-12c.

MS m/z (ESI): 394.1 [M+H⁺]

¹H NMR (400 MHz, DMSO-d₆) δ 10.94 (s, 1H), 8.72 (d, J=2.4 Hz, 1H), 8.66 (dd, J=4.7, 1.3 Hz, 1H), 8.04-7.96 (m, 1H), 7.82 (d, J=7.1 Hz, 1H), 7.59 (dd, J=8.2, 4.8 Hz, 1H), 7.47-7.37 (m, 3H), 6.53 (d, J=1.6 Hz, 1H), 6.44 (dd, J=7.1, 1.9 Hz, 1H), 4.37 (dd, J=12.3, 5.0 Hz, 1H), 2.86-2.75 (m, 1H), 2.61-2.54 (m, 1H), 2.42-2.28 (m, 1H), 2.09-2.02 (m, 1H).

### Example 26

### N-(4-(2-Chloro-3-(2,6-dioxopiperidin-3-yl)phenyl)pyridin-2-yl)cyclopropanecarboxamide (Compound I-29)

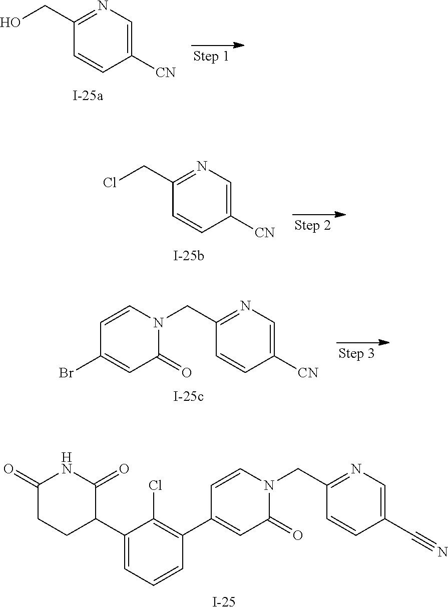

### Step 1

### N-(4-Bromopyridin-2-yl)cyclopropanecarboxamide (I-29b)

To a solution of I-29a (500 mg, 2.9 mmol) and pyridine (686 mg, 8.7 mmol) in dichloromethane (10 mL) was added cyclopropanecarbonyl chloride (363 mg, 3.5 mmol), and the mixture was stirred at 0° C. for 1 hour. After quenching with saturated ammonium chloride solution, the mixture was extracted with ethyl acetate. The organic phase was concentrated under reduced pressure and the residue was purified by silica gel column chromatography (petroleum ether/ethyl acetate=100/0 to 84/16 v/v) to give the target product I-29b (428 mg, 61%).

MS m/z (ESI): 241.0 [M+H⁺]

### Step 2

### N-(4-(2-Chloro-3-(2,6-dioxopiperidin-3-yl)phenyl)pyridin-2-yl)cyclopropanecarboxamide (I-29)

Compound I-29 was synthesized according to the procedure of Step 3 for Example 12, but I-29b was used instead of I-12c.

MS m/z (ESI): 384.1 [M+H⁺]

¹H NMR (400 MHz, DMSO-d₆) δ 10.92 (s, 2H), 8.39 (dd, J=5.1, 0.6 Hz, 1H), 8.13 (s, 1H), 7.47-7.40 (m, 2H), 7.33 (dd, J=5.3, 4.0 Hz, 1H), 7.13 (dd, J=5.1, 1.6 Hz, 1H), 4.35 (dd, J=12.3, 5.0 Hz, 1H), 2.85-2.74 (m, 1H), 2.59-2.53 (m, 1H), 2.42-2.28 (m, 1H), 2.09-1.96 (m, 2H), 0.85-0.77 (m, 4H).

### Example 27

### 3-(2-Chloro-3-(2-((6-cyclopropylpyridin-2-yl)methoxy)pyridin-4-yl)phenyl)piperidine-2,6-dione (Compound I-30)

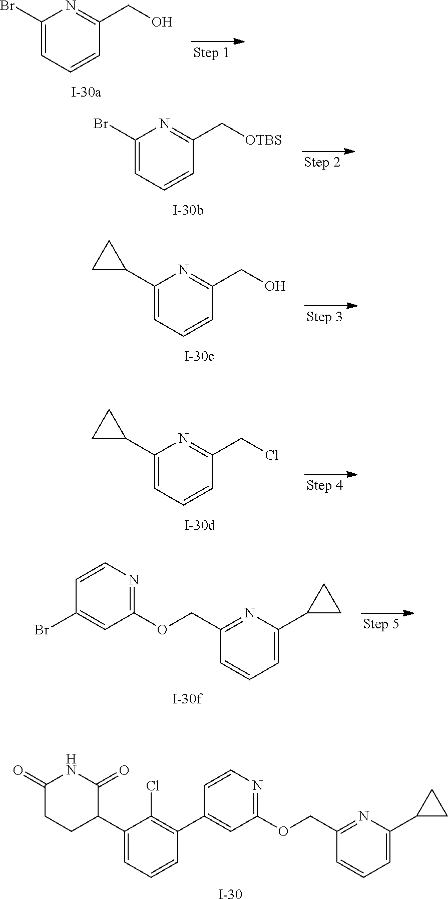

### Step 1

### 2-Bromo-6-(((tert-butyldimethylsilyl)oxy)methyl)pyridine (I-30b)

To a solution of (6-bromopyridin-2-yl)methanol I-30a (2.6 g, 14 mmol) in dichloromethane (30 mL) were added TBSCl (2.5 g, 17 mmol) and imidazole (1.4 g, 21 mmol). The reaction mixture was stirred at room temperature for 1 hour. After quenching with water, the mixture was extracted with ethyl acetate. The organic phase was dried over anhydrous sodium sulfate and concentrated under reduced pressure. The residue was purified by silica gel column chromatography (petroleum ether) to give the target product I-30b (3.5 g, 84%).

MS m/z (ESI): 302.1 [M+H⁺]

### Step 2

### (6-Cyclopropylpyridin-2-yl)methanol (I-30c)

To a solution of I-30b (1.3 g, 4.9 mmol) in THF (15 mL) was added a solution of TBAF in THF (1 M, 4.9 mL, 4.9 mmol). The reaction mixture was stirred at room temperature for 0.5 hour and concentrated under reduced pressure. The residue was purified by silica gel column chromatography (petroleum ether/ethyl acetate=100/0 to 0/100 v/v) to give the target product I-30c (0.7 g, 94%).

MS m/z (ESI): 150.1 [M+H⁺]

### Steps 3-4

### 4-Bromo-2-((6-cyclopropylpyridin-2-yl)methoxy)pyridine (1-30e)

I-30e was synthesized according to the procedures of Steps 1-2 for Example 12, but I-30c was used instead of I-12a.

MS m/z (ESI): 305.0 [M+H⁺]

### Step 5

### 3-(2-Chloro-3-(2-((6-cyclopropylpyridin-2-yl)methoxy)pyridin-4-yl)phenyl)piperidine-2,6-dione (I-30)

I-30 was synthesized according to the procedure of Step 2 for Example 10, but I-30e was used instead of I-10b.

MS m/z (ESI): 448.1 [M+H⁺]

¹H NMR (400 MHz, DMSO-d₆) δ 10.92 (s, 1H), 8.22 (d, J=5.3 Hz, 1H), 7.79-7.68 (m, 1H), 7.46-7.40 (m, 2H), 7.39-7.33 (m, 1H), 7.31-7.19 (m, 2H), 7.07 (d, J=5.2 Hz, 1H), 6.97 (s, 1H), 5.42 (s, 2H), 4.36 (dd, J=12.3, 5.0 Hz, 1H), 2.86-2.73 (m, 1H), 2.60-2.53 (m, 1H), 2.40-2.27 (m, 1H), 2.18-1.99 (m, 2H), 1.04-0.88 (m, 4H).

### Example 28

### 3-(2-Chloro-3-(1-((5-cyclopropylpyridin-2-yl)methyl)-6-oxo-1,6-dihydropyridazin-4-yl)phenyl)piperidine-2,6-dione (Compound I-31)

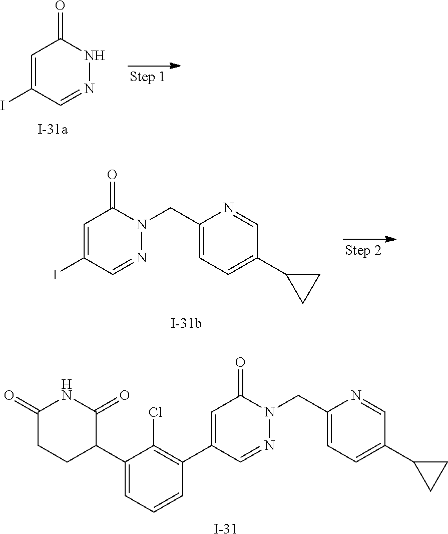

### Step 1

### 2-((5-Cyclopropylpyridin-2-yl)methyl)-5-iodopyridazin-3(2H)-one (1-31b)

To a solution of I-31a (500 mg, 2.3 mmol) and I-6c (453 mg, 2.7 mmol) in DMSO (10 mL) was added cesium carbonate (1.1 g, 3.4 mmol), and the reaction mixture was stirred at 60° C. for 1 hour. The mixture was added with water and extracted with ethyl acetate. The organic phase was concentrated under reduced pressure and the residue was purified by silica gel column chromatography (petroleum ether/ethyl acetate=100/0 to 36/64 v/v) to afford the target product I-31b (400 mg, 50%).

MS m/z (ESI): 354.1 [M+H⁺]

### Step 2

### 3-(2-Chloro-3-(1-((5-cyclopropylpyridin-2-yl)methyl)-6-oxo-1,6-dihydropyridazin-4-yl)phenyl)piperidine-2,6-dione (I-31)

I-31 was synthesized according to the procedure of Step 3 for Example 12, but I-31b was used instead of I-12c.

MS m/z (ESI): 449.1 [M+H⁺]

¹H NMR (400 MHz, DMSO-d₆) δ 10.94 (s, 1H), 8.34 (d, J=2.1 Hz, 1H), 8.05 (d, J=2.2 Hz, 1H), 7.53-7.45 (m, 3H), 7.42 (dd, J=8.1, 2.3 Hz, 1H), 7.18 (d, J=8.1 Hz, 1H), 7.03 (d, J=2.2 Hz, 1H), 5.35 (s, 2H), 4.37 (dd, J=12.3, 5.0 Hz, 1H), 2.87-2.75 (m, 1H), 2.61-2.53 (m, 1H), 2.40-2.26 (m, 1H), 2.08-1.90 (m, 2H), 1.02-0.95 (m, 2H), 0.75-0.68 (m, 2H).

### Example 29

### (S)-3-(2-Chloro-3-(1-((5-cyclopropylpyridin-2-yl)methyl)-2-oxo-1,2-dihydropyridin-4-yl)phenyl)piperidine-2,6-dione (I-32)

### (R)-3-(2-Chloro-3-(1-((5-cyclopropylpyridin-2-yl)methyl)-2-oxo-1,2-dihydropyridin-4-yl)phenyl)piperidine-2,6-dione (I-33)

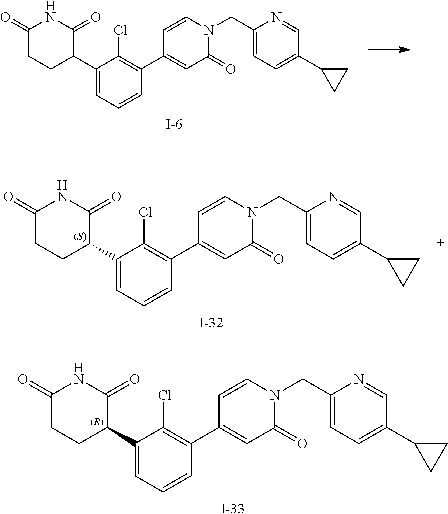

Compound I-6 (300 mg) was separated by chiral preparation (chiral column: CHIRALPAK IA, 2.5 cm I.D.×25 cm L, 10 μm; mobile phase: ethanol/ethyl acetate=65/35 v/v; flow rate: 50 mL/min; temperature: 40° C.) to afford enantiomers which were further purified by prep-HPLC to yield the target products I-32 (92 mg, 31%) and I-33 (70 mg, 23%).

Characterization data for I-32:

MS m/z (ESI): 448.1 [M+H⁺]

¹H NMR (400 MHz, DMSO-d₆) δ 10.92 (s, 1H), 8.36 (d, J=2.1 Hz, 1H), 7.83 (d, J=7.0 Hz, 1H), 7.48-7.31 (m, 4H), 7.20 (d, J=8.1 Hz, 1H), 6.37 (d, J=1.8 Hz, 1H), 6.31 (dd, J=7.0, 1.9 Hz, 1H), 5.16 (s, 2H), 4.35 (dd, J=12.2, 5.0 Hz, 1H), 2.85-2.71 (m, 1H), 2.62-2.53 (m, 1H), 2.39-2.25 (m, 1H), 2.09-1.89 (m, 2H), 1.04-0.95 (m, 2H), 0.77-0.67 (m, 2H).

Characterization data for I-33:

MS m/z (ESI): 448.1 [M+H⁺]

¹H NMR (400 MHz, DMSO-d₆) δ 10.92 (s, 1H), 8.36 (d, J=2.1 Hz, 1H), 7.83 (d, J=7.0 Hz, 1H), 7.48-7.32 (m, 4H), 7.20 (d, J=8.1 Hz, 1H), 6.37 (d, J=1.8 Hz, 1H), 6.31 (dd, J=7.0, 1.9 Hz, 1H), 5.16 (s, 2H), 4.35 (dd, J=12.2, 5.0 Hz, 1H), 2.87-2.72 (m, 1H), 2.62-2.53 (m, 1H), 2.39-2.25 (m, 1H), 2.09-1.88 (m, 2H), 1.04-0.95 (m, 2H), 0.76-0.68 (m, 2H).

### Biology Experiments

### Example 30

### Determination of VAV1 Degradation

The test compound was dissolved in DMSO and diluted to 2 mM, followed by a 5-fold serial dilution with DMSO. Each concentration point was further diluted by 50-fold with RPMI1640 medium (Thermo Fisher, catalog number 72400-047). If the compound had a low DC₅₀ value, the starting concentration of the compound was further reduced.

Jurkat VAV1-HiBiT cells were obtained by overexpressing VAV1 fused with HiBiT at C-terminus in Jutkat E6.1 cells (Nanjing Cobioer, catalog number CBP60942). The cells were cultured in RPMI1640 complete medium [which contained 10% FBS (ExCell, catalog number FSP500)]. Cells (300,000 cells/mL) were seeded in 27 μL of complete medium in a 384-well plate, with 3 μL of compound solution added per well, and incubated at 37° C. in a 5% CO₂ incubator for 24 hours. The cell culture plate was equilibrated to room temperature, and then processed according to instruction from the Nano-GloHiBiT Lytic System kit (Promega, catalog number N3030), by adding an equal volume of Nano-Glo reagent for complete lysis. After standing at room temperature for 20 minutes, the luminescence signal was read using a microplate reader (EnVision, Perkin Elmer). The group containing 0.2% DMSO was used as a non-VAV1 degradation control and the group containing medium only was used as the background control. % degradation=1−(luminescence signal_{compound}−luminescence signal_{background})/(luminescence signal_{DMSO}−luminescence signal_{background})×100%. The compound induced VAV1 degradation curve was plotted using XLfit software (ID Business Solutions Ltd., UK) to calculate its DC₅₀ value. The experimental results are shown in Table 1.

### Example 31

### Determination of Inhibition of IL-2 Activation Induced by CD3/CD28

The test compound was dissolved in DMSO and diluted to 2 or 5 mM, followed by a 5-fold serial dilution with DMSO. Each concentration point was further diluted by 62.5-fold with RPMI1640 medium (ThermoFisher, catalog number 72400-047). If the compound's IC₅₀ value was low, the starting concentration of the compound was further reduced.

Jurkat IL-2-Luc cells (BPS Bioscience, catalog number 60481) were cultured at 37° C. in RPMI1640 medium with addition of a mixture of 10% FBS (ExCell Bio, catalog number FSP500) and 100 units/mL penicillin-streptomycin (ThermoFisher, catalog number 15140122). The cells were seeded into 384-well plates (ThermoFisher, catalog number 164610) containing 30 μL RPMI1640 medium at a density of 1×10⁶ cells/mL per well. After overnight incubation at 37° C., 5 μL of the test compound solution was added to each well, followed by the addition of 5 μL of antibody mixture 30 minutes later [containing a final concentration of 10 μg/mL CD3 antibody (ThermoFisher, catalog number 16-0037-85) and 5 [g/mL CD28 antibody (ThermoFisher, catalog number 16-0289-85)]. The cells were further incubated for 6 hours at 37° C. in a 500 CO₂ incubator, then processed according to instructions from the ONE-Glo luciferase assay kit (Promega, catalog number E6120). The cell culture plate was equilibrated to room temperature, 20 μL of ONE-Glo reagent was added and mixed thoroughly, and after a 10-minute incubation at room temperature, the luminescence signal at 560 nm emission wavelength was read using a plate reader (EnVision, Perkin Elmer).

In this experiment, the 000 inhibition group was one added with a mixture of CD3 and CD28 antibodies for stimulation, but no compound added. The 10000 inhibition group was one without addition of a mixture of antibodies and the test compound. 00 inhibition=100−100*(luminescence signal_{compound}−luminescence signal_{100% inhibition}/(luminescence signal_{0% inhibition}−luminescence signal_{1000% inhibition}). The XLfit software was used to plot the inhibition curve of the test compound on CD3/CD28 induced IL-2 activation and calculate the corresponding IC₅₀ values. The experimental results are shown in Table 1.

TABLE 1

| | | | Inhibition of |
|---|---|---|---|
| | | | CD3/CD28 |
| | VAV1 | VAV1 | induced IL-2 |
| Compound | Degradation | Degradation | activation |
| Number | DC₅₀ (nM) | Dₘₐₓ (%) | IC₅₀ (nM) |
| I-1 | >2000 | | |
| I-2 | 13 | 95 | 23 |
| I-3 | 395 | 47 | |
| I-4 | 21 | 95 | |
| I-5 | 33 | 85 | |
| I-6 | 2.4 | 98 | 18 |
| I-7 | >2000 | | |
| I-8 | 211 | 40 | |
| I-9 | >2000 | | |
| I-10 | 113 | 33 | |
| I-11 | >2000 | | |
| I-12 | 4.6 | 98 | 17 |
| I-13 | >2000 | | |
| I-14 | 6.8 | 98 | 14 |
| I-15 | >2000 | | |
| I-16 | 16 | 99 | |
| I-17 | 13 | 96 | |
| I-18 | 16 | 97 | |
| I-19 | 9.2 | 98 | |
| I-20 | 2.6 | 98 | 10 |
| I-21 | 2.9 | 99 | |
| I-22 | 76 | 61 | |
| I-23 | 157 | 86 | |
| I-24 | 169 | 64 | |
| I-25 | 45 | 97 | |
| I-26 | 9.4 | 97 | 17 |
| I-27 | 41 | 97 | |
| I-28 | >2000 | | |
| I-29 | >2000 | | |
| I-30 | 296 | 73 | |
| I-31 | 9.9 | 97 | |
| I-32 | 1.6 | 99 | 8.7 |
| I-33 | 24 | 98 | |

### Example 32

### In Vivo Efficacy Study in MOG₃₅₋₅₅-Induced EAE Model in Female C57BL/6 Mice

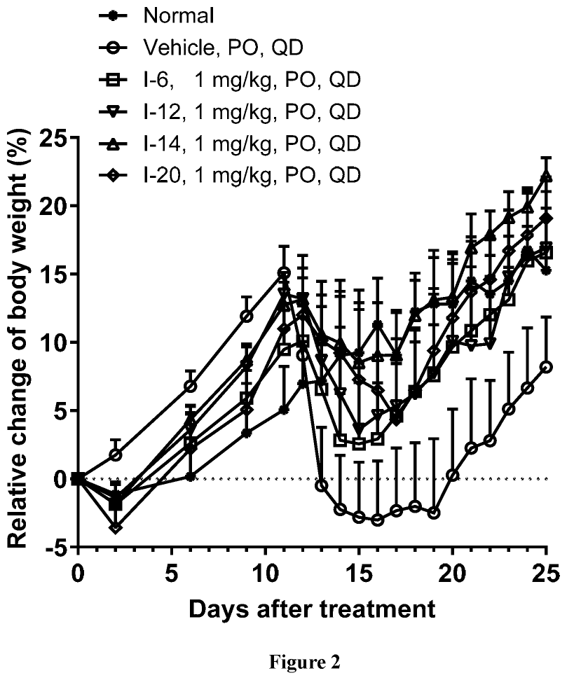

Female C57BL/6 mice (6-8 weeks old) were purchased from Beijing Vital River Laboratory Animal Technology Co., Ltd and were acclimatized at least one week prior to immunization. Desiccated M. tuberculosis H37Ra (BD, #231141) was suspended in incomplete Freund's Adjuvant (IFA, Merck, #F5506) to a final concentration of 8 mg/mL, and then mixed with an equal volume of a MOG₃₅₋₅₅ peptide solution (3 mg/mL in PBS). The resulting mixture was thoroughly homogenized to an emulsion on ice using a homogenizer. Each mouse was immunized with 100 μL of the emulsion via subcutaneous injection at the back and flank (200 μL in total). Pertussis toxin (500 ng per mouse) in 200 μL of PBS was intraperitoneally administered on the day of immunization and 48 hours later. Compounds 1-6, 1-12, 1-14, and I-20 were dissolved in 10% (w/v) captisol and orally administered daily at 1 mg/kg starting from the immunization day. The clinical symptom was evaluated based on the following scoring system:
EAE clinical symptoms and scoring:
0: No obvious change;
0.5: Tip of tail is limp;
1.0: Limp tail;
1.5: Limp tail and hind leg inhibition;
2: Limp tail and weakness of hind legs;
2.5: Limp tail and dragging of hind legs;
3.0: Limp tail and complete paralysis of hind legs (most common);
3.5: Limp tail and complete paralysis of hind legs;
4.0: Limp tail, complete hind leg and partial front leg paralysis;
4.5: Complete hind and partial front leg paralysis;
5.0: Moribund. FIG. 2 shows that daily oral dosing of compounds I-6, I-12, I-14, and I-20 at 1 mg/kg did not

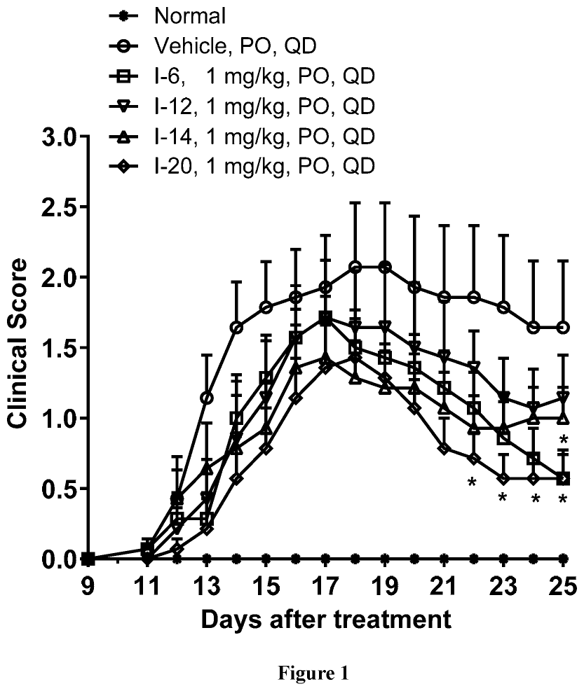

Weight loss was also monitored as an indicator of disease severity. Clinical score and body weight were recorded daily during the treatment. The results showed that daily oral dosing of compounds I-6, I-12, I-14, and I-20 at 1 mg/kg significantly alleviated EAE progression with less clinical scores (FIG. 1) and did not have significant weight loss (FIG. 2) in the treatment groups compared to those in the vehicle group.

What is claimed is:

1. A compound of Formula (II), or its pharmaceutically acceptable salts, stable isotope derivatives and stereoisomers, which is a compound of Formula II: 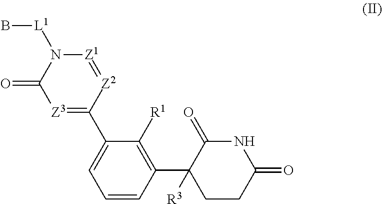
wherein:
B is 5- to 6-membered heteroaryl, phenyl or 4- to 10-membered heterocyclyl, where one or more hydrogens of the heteroaryl, phenyl and heterocyclyl are optionally substituted by R⁵;
Z¹, Z² and Z³ are each independently CR^{4′} or N;
L¹ is —C₁₋₆ alkylene-, bond, *—C₂₋₆ alkylene-O—** or *—C₁₋₆ alkylene-NR′—**, where * indicates L¹ connected to the N atom of the heteroaryl ring, ** indicates L¹ connected to B, and one or more hydrogens of the alkylene are optionally substituted by D, halogen or C₁₋₆ alkyl;
R³ is H, D, halogen, C₁₋₆ alkyl or fluorinated C₁₋₆ alkyl;
R^{4′} is H or R⁴;
R⁴ and R⁵ are each independently halogen, cyano, oxo, —OR′, —NR′R″, —COOH, —C(O)NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl; and
R′ and R″ are each independently H, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OC₁₋₆ alkyl or C₁₋₆ alkyl.

2. The compound according to claim 1, or its pharmaceutically acceptable salts, stable isotope derivatives and stereoisomers, which is a compound of Formula III: 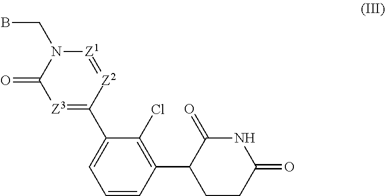
wherein:
B is 5- to 6-membered heteroaryl containing 1-3 heteroatoms of nitrogen, oxygen and sulfur, where one or more hydrogens of the heteroaryl are optionally substituted by R⁵;
Z¹, Z² and Z³ are each independently CH or N; and
R⁵ is halogen, cyano, C₁₋₆ alkyl, C₃₋₈ cycloalkyl, —OC₁₋₆ alkyl or —OC₃₋₈ cycloalkyl, where one or more hydrogens of the alkyl are optionally substituted by F.
